(19) United States
(12) Patent Application Publication
(10) Pub. No.: US 2026/0294440 A1
(43) Pub. Date: Oct. 1, 2026

(54) LASER CUT CATHETERS FOR VASCULAR EMBOLIZATION

(71) Applicant: Q'APEL MEDICAL, INC., Fremont, CA (US)

(72) Inventors: Michael TUROVSKIY, San Leandro, CA (US); Kip WETTER, San Francisco, CA (US); Jeffery KROLIK, Campbell, CA (US); Robert Ferrara, Cold Springs Harbor, NY (US)

(21) Appl. No.: 19/187,805

(22) Filed: Apr. 23, 2025

Related U.S. Application Data
(60) Provisional application No. 63/777,590, filed on Mar. 25, 2025.

(51) Int. Cl. A61B 17/12; A61M 25/00; A61M 25/09

(52) U.S. Cl. CPC A61B 17/12186; A61B 17/12031; A61B 17/12109; A61M 25/0045; A61M 25/0054; A61M 25/008; A61M 25/09; A61B 2017/1205; A61M 2025/0004; A61M 2025/0081

(57) ABSTRACT

Provided herein are devices and methods having an assembly of catheters that may be configured for use in delivering a composition in a subject. The devices and methods may include interior catheters configured to fit within an exterior catheter, wherein the interior catheter may have a laser cut tube (LCT) construction. In some embodiments, the interior catheter is advanced through the exterior catheter to a target site to deliver the composition. In some embodiments, the exterior catheter is configured to preserve a position proximal to one or more delivery sites, thus reducing time required to reach the target site(s) and/or for providing a faster treatment with improved handling.

### CROSS-REFERENCE

This application claims the benefit of U.S. Provisional Application No. 63/777,590, filed Mar. 25, 2025, which is incorporated herein by reference in its entirety.

### BACKGROUND

Chronic subdural hematoma (SDH) is a condition where the blood vessels on the outside surface of the brain bleed and cause a collection of blood between the surface of the brain and its outer covering (dura). SDH may cause compression of the brain due to the pressure applied by the collection of blood. In serious cases, SDH may lead brain damage or death. Thus, it is important to treat SDH quickly. Usually, SDH is treated by a surgery, including but not limited to a craniotomy or creation of a burr hole to allow drainage of the collection of blood. While these surgical interventions may treat the SDH, these interventions often do not address the underlying cause of the brain bleed, and the patients are likely to experience a recurrence of the SDH.

### SUMMARY

Individuals may develop a hematoma from a blood vessel may present a significant health issue that may require a treatment to remove the collection of blood and/or treat the underlying blood vessel to prevent further bleeding. When a hematoma occurs in the subdural space, an individual may suffer compression of the brain due to the pressure of the blood, which may lead to brain damage or death. Conventional treatment is typically surgical, where a craniotomy or burr hole is performed to allow drainage of the collection of blood. This may provide immediate treatment of the hematoma, but may not solve the underlying issue that caused the bleed and result in a recurrence of the hematoma.

Catheters are often used to reach and provide a treatment in a difficult-to-reach target site of body of an individual. Depending on the anatomy and the treatment, a catheter assembly configured to reach one or more treatment sites may allow for reducing the time required to reach the target site(s) and/or for providing a faster treatment with improved handling. In some applications, such as but not limited to subdural hematoma, where a blood vessel leaks and causes a collection of blood between the brain and the dura mater, the ability to quickly reach the target site(s) and provide a lasting treatment (e.g., stop bleeding from the blood vessel(s)) may be critical to the success of the treatment. The individual may have improved outcomes as measured by improved neurological and functional outcomes (e.g., decreased compression of the brain) and/or reduced procedural complications (e.g., hemorrhage, perforation).

As such, it would be highly beneficial to be able to easily prepare a catheter assembly configured to reach one or more treatment sites, comprising a catheter having variable flexibility and/or stiffness along its longitudinal length tuned for its use. For example, a catheter having a lower stiffness and/or higher flexibility at a distal portion and a higher stiffness and/or lower flexibility at a proximal portion may allow for an improved maneuverability and handleability. Such a catheter may be beneficial in reaching an artery in the brain with a tortuous path and/or providing a treatment for neurovascular applications.

Provided herein are catheter systems for embolizing a middle meningeal artery (MMA), the system comprising: an exterior catheter comprising a laser cut tube (LCT) having a lumen; and an interior catheter comprising a LCT, the interior catheter configured to extend within the lumen of the exterior catheter, wherein the exterior catheter has an outer diameter at a distal portion of at most about 0.04 inch, wherein the interior catheter has an outer diameter at a distal portion of at most about 0.03 inch, and wherein a ratio of the outer diameter at the distal portion of the interior catheter to an outer diameter of a proximal portion of the interior catheter is between about 0.8 and about 1.2. In some embodiments, the outer diameter of the interior catheter varies by at most 10% longitudinally. In some embodiments, the system comprises a guidewire configured to extend within a lumen of the interior catheter. In some embodiments, the interior catheter has a wall thickness that is substantially constant. In some embodiments, the interior catheter is pliant. In some embodiments, the interior catheter comprises a pliant distal tip. the interior catheter comprises a detachable distal tip. In some embodiments, at least one of the LCT of the interior catheter and the LCT of the exterior catheter comprises a first section comprising a plurality of first cuts and a cut filler comprising a polymer that is filling a first cut of the plurality of the first cut. In some embodiments, at least one of the LCT of the interior catheter and the LCT of the exterior catheter comprises a second section adjacent to the first section, the second section comprising a plurality of second cuts, and a cut filler comprising a polymer that is filling a second cut of the plurality of second cuts, wherein the first cut of the plurality of first cuts has an average first kerf that is wider than an average kerf of the second cut of the plurality of second cuts. In some embodiments, at least one of the interior catheter and the exterior catheter comprises an outer surface. In some embodiments, the catheter system further comprises an outer jacket configured to cover at least one of the outer surface of the interior catheter and the outer surface of the exterior catheter. In some embodiments, the outer jacket is configured to contact the cut filler, to bond to the cut filler, or any combination thereof. In some embodiments, wherein the catheter system comprises a liner covering at least one of a lumen of the interior catheter and the lumen of the exterior catheter. In some embodiments, the liner comprises a polymer, wherein a polymer comprises polyethylene, PTFE, or poly(ether-block-amide), or a combination thereof. In some embodiments, the liner is configured to contact the cut filler, to bond to the cut filler, or any combination thereof. In some embodiments, an embolic composition comprises a liquid embolic, a particle, or a coil, or a combination thereof.

Provided herein are catheter systems for embolizing a middle meningeal artery (MMA), the system comprising: an exterior catheter comprising a laser cut tube (LCT) having a lumen; and an interior catheter comprising a LCT having a lumen, the interior catheter configured to extend within the lumen of the exterior catheter, wherein the exterior catheter is configured to fit within the MMA, wherein the outer diameter of the interior catheter varies at most by 10% longitudinally.

Provided herein are methods of delivering an embolic composition to a blood vessel, the method comprising: (a) advancing an exterior catheter through the vasculature proximal to a target site; (b) advancing an interior catheter comprising a laser cut tube (LCT) through a lumen of the exterior catheter to the target site, wherein a distal tip of the interior catheter extends distally from a distal end of the exterior catheter; and (c) delivering the embolic composition through a lumen of the interior catheter to the target site. In some embodiments, wherein the method further comprises: (d) withdrawing the interior catheter from the lumen of the exterior catheter; (e) advancing the exterior catheter to a second target site; and (f) delivering the embolic composition through the lumen of the exterior catheter to the second target site. In some embodiments, the method further comprises: (d) withdrawing the interior catheter from the lumen of the exterior catheter; (e) advancing the exterior catheter proximal to a second target site; (f) advancing a second interior catheter through the lumen of the exterior catheter to a second target site, wherein a distal tip of the second interior catheter extends distally from the distal end of the exterior catheter; and (g) delivering the embolic composition through a lumen of the second interior catheter to the second target site.

### BRIEF DESCRIPTION OF THE DRAWINGS

The novel features of the technology are set forth with particularity in the appended claims. A better understanding of the features and advantages of the present technology will be obtained by reference to the following detailed description that sets forth illustrative embodiments, in which the principles of the technology are utilized, and the accompanying drawings (also “Figure” and “FIG.” herein), of which:

FIG. 1 illustrates anatomy of vasculature of the dura, including the middle meningeal artery (MMA), and corresponding vasculature.

FIGS. 2A-2D illustrates an example of a catheter assembly. FIG. 2A illustrates a nested catheter assembly. FIG. 2B illustrates a cross section of an interior catheter having an inner diameter, an outer diameter, and a wall thickness. FIG. 2C illustrates a cross section of an exterior catheter having an inner diameter, an outer diameter, and a wall thickness. FIG. 2D illustrates a cross section of an interior catheter positioned in an exterior catheter, forming a gap.

FIGS. 3A-3I illustrate exemplary procedures performed with exemplary embodiments of a catheter assembly. FIG. 3A illustrates an access catheter positioned in a vessel. FIG. 3B illustrates an exterior catheter advanced through an access catheter. FIG. 3C illustrates an interior catheter advanced through an exterior catheter. FIG. 3D illustrates an occlusive cast delivered to a first target site by an interior catheter. FIG. 3E illustrates a second interior catheter advanced through an exterior catheter to a second target site. FIG. 3F illustrates an occlusive cast delivered to a second target site by a second interior catheter. FIG. 3G illustrates an outer catheter positioned at a second target site. FIG. 3H illustrates an occlusive cast delivered to a second target site by an exterior catheter. FIG. 3I illustrates occlusive casts delivered to target sites.

FIGS. 4A-4B illustrate exemplary embodiments of a catheter.

FIG. 5 illustrates an example of a cut tube with an interrupted helical cut pattern.

FIGS. 6A-6D illustrate example of stretched cut tube sections with varying degrees of stretch.

FIGS. 7A-7B illustrate example of a cut tube with a stiffer section and a more flexible section.

FIGS. 8A-8B illustrate example of a catheter with an outer jacket.

### DETAILED DESCRIPTION

A hematoma from a blood vessel may present a significant health issue that may require a treatment to remove the collection of blood and/or treat the underlying blood vessel to prevent further bleeding. When a hematoma occurs in the subdural space as in chronic subdural hematoma (SDH), an individual may suffer compression of the brain due to the pressure of the blood, which may lead to brain damage or death. One approach to treating hematoma is embolization where the vessels most commonly associated with the bleeds that cause hematoma are blocked or closed off. For SDH, the middle meningeal artery (MMA) is often a common site for a leaky vessel and for embolization. In embolization, a material may be delivered into the vessel to obstruct the blood flow. Often, the goal of the embolization is to stop blood flow and eliminate blood pressure within the vessel so that the bleeds causing hematoma are reduced. MMA embolization has been shown to safe and effective technique in treating SDH and reducing the recurrence of SDH.

The materials used in embolization may include but are not limited to particles, coils, or a liquid embolic. Often, particles comprise a PVA sponge material that may be injected into the target vessels to occlude blood flow. Coils may be used to block flow at their deployment location. The challenge with particles and coils is that they tend to create a more proximal occlusion, but do not affect the vasculature more distally. In some embodiments, if there is collateral circulation, or interconnection to vessels that have not been occluded, the bleeds may not be effectively eliminated.

Liquid embolics have been used to address these challenges. Liquid embolics may be injected as a liquid to the target site and flow to more distal vessels before solidifying. In some embodiments, the materials used in embolization are typically delivered to the target site using a catheter. The solidified liquid embolic that has taken the shape of the target vasculature is commonly called a cast, because it forms a solid in the shape of the vessel being occluded. Several liquid embolics have been used in MMA embolization, including but not limited to Squid (Balt Group), Phil (Terumo Neuro), Truefill (Cerenovus), and Onyx (Medtronic).

There is a challenge with the delivery of liquid embolics. Often, the injections for embolization may be performed in several target sites, such as several branches (or pedicles) of the neurovasculature, through a catheter placed in the desired deployment location. During the delivery, the liquid embolic may coat the internal diameter of the delivery catheter. In some cases, the liquid embolic solidifies upon exposure to blood flow, and the solidification may make subsequent injections through the catheter unreliable. In some cases, navigation of the catheters is commonly performed with a guidewire in the central lumen. In some cases, if liquid embolic is present, the liquid embolic may coat the guidewire and may make its performance unreliable or problematic. In some cases, as the catheter is repositioned, there is a risk of the liquid embolic being released into a blood vessel where it is not intended. In some cases, the unintended release of the liquid embolic may have undesirable effect. For example, the unintended release of the liquid embolic to a branch of the ophthalmic artery may cause impairment or loss of vision. Because of the challenges arising during liquid embolic delivery with traditional catheter assemblies, a single injection may be performed through a delivery catheter, which is removed and replaced with a new catheter for subsequent injections. In some cases, the capability to perform a first injection through a first catheter and remove and replace that catheter with a second catheter for a second injection may provide advantages in safety and accuracy of an embolization procedure. While the catheter may be replaced after each liquid embolic delivery, this creates a time lag in placing a new catheter into place for the subsequent liquid embolic delivery. In some cases, the vascular path to the target site may include tortuosity that makes the navigation of the catheters difficult to the next target site. As such, provided herein are systems comprising two or more catheters that are telescoped within another catheter for embolization and methods of accessing multiple target sites using such system. In some embodiments, the assembly comprises three or more telescoping catheters. In some embodiments, the catheter assembly is sufficiently flexible to bend as the catheter assembly is advanced through a tortuous anatomy. In some embodiments, the catheter assembly is sufficiently flexible to advance through tortuous anatomy at least in part because of a laser cut tube (LCT) assembly. In some cases, the capability to perform a first injection through a first catheter and remove and replace that catheter with a second catheter for a second injection may provide advantages in safety and accuracy of an embolization procedure. In some cases, an interior catheter (also referred herein as inner catheter) of the system may be removed after delivering a liquid embolic to a first target site, when the inner catheter may have a solidified liquid embolic in its lumen, and an exterior catheter (also referred herein as outer catheter), with or without another inner catheter may access a second target site. Such catheter systems may be possible by using laser cut catheters. In some cases, using LCTs in the catheter system allows for thin-walled, small diameter catheters that can be stacked and maintain maneuverability through a tortuous vessel. The catheter assembly configured to reach one or more treatment sites as provided herein may allow for reducing the time to time required to reach the target site(s) and/or for providing a faster treatment with improved handling. The capability to easily prepare such a catheter assembly configured to reach one or more treatment sites, comprising a catheter having variable flexibility and/or stiffness along its longitudinal length tuned for its use would be helpful. Provided herein are catheter assemblies comprising an interior catheter configured to fit inside an exterior catheter and methods of using such catheters to deliver a therapeutic to at least one target site. In some embodiments, the filler material at least partially extends through the cuts. In some embodiments, the filler material fills at least a portion of the plurality of cuts In some embodiments, the filler material sets the cuts in the stretched position in the stretched portions, thereby preparing a portion of the tube having a different mechanical property (e.g., flexibility, stiffness) from a portion that is applied with a different amount of stretch, including no stretch.

### I. Overview

In order to reach and provide treatment in a difficult-to-reach target sit of a body of an individual, catheters are commonly used to reach and treat the target site. Depending on the anatomy to the target site and the treatment, a catheter assembly configured to preserve a position proximal to the one or more treatment sites may be valuable. In some instances, the preserved position may help to for reduce the time required to reach the target site(s), provide a faster treatment time, and/or reduce the amount of radiation the individual may be exposed to. The individual may have improved outcomes as measured by improved neurological and functional outcomes (e.g., decreased compression of the brain) and/or reduced procedural complications (e.g., hemorrhage, perforation).

Usually, the use of catheters for treatment for neurovascular applications involves navigating through tortuous paths of arteries in the head and neck. Blood vessel abnormalities, including but not limited to hematomas, arteriovenous malformations (AVMs), and aneurysms, may be located throughout the neurovasculature of the body. Sometimes, these blood vessel abnormalities present a significant health issue that may require treatment. For example, a subdural hematoma (SDH), which is a collection of blood accumulated in the subdural space, may cause compression of the brain due to the pressure of the blood in the subdural space. In these instances, the compression may lead to brain damage or death. SDHs and/or the vessel causing the SDH may occur in any location in the subdural space (between the dura mater and the brain surface) and may be widespread. Sometimes, SDH may be chronic subdural hematomas (cSDH). In these instances, it is often necessary to treat the underlying issue causing the SDH in order to help prevent a recurrence of the SDH. In some cases, obstructing flow of one or more vessels associated with the bleeds causing the SDH can treat the SDH.

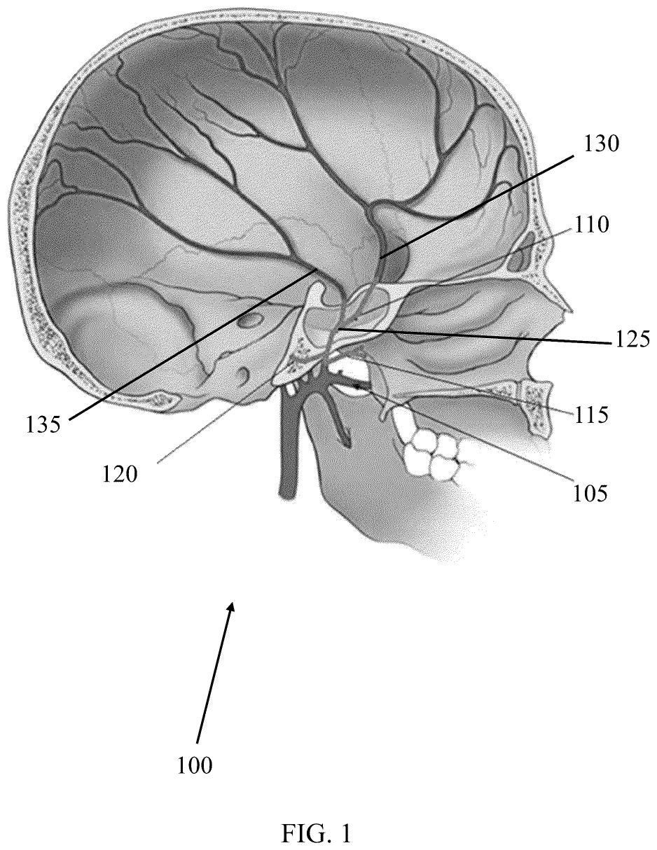

A non-limiting example of a cause of a SDH may be a bleed from the Middle Meningeal Artery (MMA). FIG. 1 illustrates typical anatomy 100 of the MMA 110 and connected vasculature. An extracranial part (EC) of the MMA 110 runs from the origin of the MMA up and medially reaching an external surface of the skull base. A diameter of the EC MMA of an adult subject may be between about 1.42 mm to about 3.98 mm. An intraosseous part (IO) of the MMA 110 extends between an external and an internal orifice of a spinous foramen of a greater wing of a sphenoid bone. A diameter of the IO MMA of an adult subject may be between about 1.34 mm to about 3.53 mm. An intracranial segment (IC) of the MMA lies within the skull after entering through the foramen spinosum and may give rise to most branches of the MMA 110. A diameter of the IC MMA of an adult subject may be between about 1.31 mm to about 3.38 mm. The MMA 110 typically arises from the internal maxillary artery (IMAX) 105 and has basal two branches before (i.e., proximal to) the MMA bifurcation 125—the petrosal branch 120, and the cavernous branch 115; the petrosal branch 120 helps supply the facial nerve, trigeminal ganglion, and the greater petrosal nerve, while the cavernous branch 115 connects the MMA 110 with the inferolateral trunk and supplies cranial nerves of the cavernous sinus and Meckel cave. At the MMA bifurcation 125, the MMA 110 divides into a frontal division 130 and a posterior division 135, wherein each division has additional branches of the MMA. In instances where a bleed from the MMA 110 causes a SDH, treatment may include an embolic delivered by a delivery catheter to a target site(s) of the MMA 110 to stop the bleeds causing the SDH. Sometimes, an access catheter is advanced up to near the origin of the MMA 110 from the IMAX 105, and the delivery catheter is advanced through the access catheter, past the origin of the MMA 110 and to the target site. Sometimes, the embolic is a liquid embolic, which is often injected in a liquid state and flows to a more distal vessel(s) before solidifying in the shape of a target vasculature (i.e., forms a “cast” in the shape of the vessel). However, each delivery of a liquid embolic will coat an internal diameter of the delivery catheter and subsequent injections through that delivery catheter are unreliable and/or unusable. Often, multiple branches of the MMA 110 require treatment to solve the SDH. In instances like these, extreme caution must be exercised to only deliver the embolic to the target site(s) to avoid liquid embolic being delivered to unintended vessels or branches, as obstructing blood flow of unintended vessels may increase complications. Thus, delivery catheters must be removed from the individual after each liquid embolic delivery and replaced with a new delivery catheter for each subsequent delivery to a new target site. In these instances, replacing a delivery catheter may require duplicating the effort of navigating the new delivery catheter through tortuous anatomy to a target site. So, a catheter assembly configured to preserve a position proximal to the one or more treatment sites may be valuable.

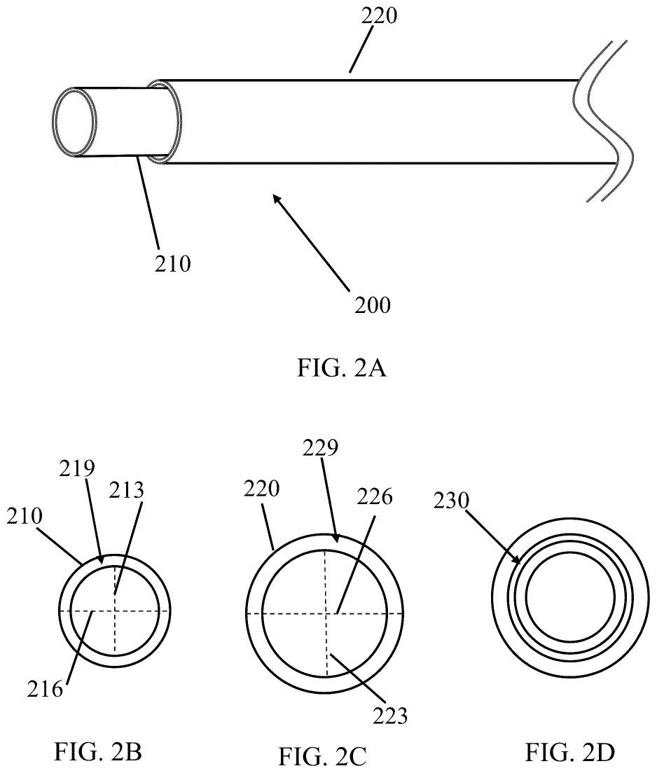

FIG. 2A-D illustrate embodiments of a catheter assembly 200. In some embodiments, an interior catheter 210 is configured to fit within an exterior catheter 220. In some embodiments, the interior catheter 210 comprises an inner diameter 213. In some embodiments, the interior catheter comprises an outer diameter 216. In some embodiments, the interior catheter comprises a wall thickness 219. In some embodiments, the exterior catheter 220 comprises an inner diameter 223. In some embodiments, the exterior catheter 220 comprises an outer diameter 226. In some embodiments, the exterior catheter 220 comprises a wall thickness 229. In some embodiments, the interior catheter 210 is configured to fit within the exterior catheter 220 such that a gap 230 is formed. In some embodiments, the gap 230 is configured to provide free movement between the interior catheter 210 and the exterior catheter 220.

In some embodiments, the assembly comprises three or more catheters that are telescoped within another catheter for embolization and methods of accessing multiple target sites using such system. In some embodiments, the catheter assembly is sufficiently flexible to bend as the catheter assembly is advanced through a tortuous anatomy. In some embodiments, the catheter assembly is sufficiently flexible to advance through tortuous anatomy at least in part because of a laser cut tube (LCT) assembly. In some embodiments, the assembly is configured to be used with a guidewire. In some embodiments, the guidewire is used to help the system navigate through tortuous anatomy to a target site.

### Interior Catheter

The catheter assembly and methods provided herein comprise a first catheter (e. g, interior catheter) and a second catheter (e.g., exterior catheter). In some embodiments, the interior catheter comprises a laser cut tube (LCT). In some embodiments, the LCT comprises a first section comprising a plurality of first cuts. In some embodiments, the LCT comprises a second section adjacent to the first section, the second section comprising a plurality of second cut. In some embodiments, the LCT comprises a cut filler comprising a polymer that is filling at least one of a first cut of the plurality of the first cut and a second cut of the plurality of second cuts. In some embodiments, the first catheter is configured to fit inside the second catheter. In some embodiments, the catheter is configured to navigate through tortuous vessels. In some embodiments, the catheter is configured to navigate through tortuous neurovasculature. In some embodiments, the catheter is configured to navigate through a middle meningeal artery (MMA). In some embodiments, the catheter is configured to navigate through a distal branch of the MMA.

In some embodiments, the catheter comprises an inner diameter, an outer diameter, and a wall thickness. In some embodiments, the outer diameter comprises a proximal outer diameter. In some embodiments, the proximal outer diameter is defined by measuring the diameter of the interior catheter at a proximal end of the interior catheter. In some embodiments, the outer diameter comprises a distal outer diameter. In some embodiments, the distal outer diameter is defined by measuring the diameter of the interior catheter at a distal end of the interior catheter. In some embodiments, a ratio of the proximal outer diameter to the distal outer diameter is less than about 1.5. In some embodiments, the proximal outer diameter and the distal outer diameter are substantially equal. In some embodiments, the inner diameter is substantially constant through the length of the catheter. In some embodiments, the inner diameter varies less than about 30%, 20%, 10%, or 5% over length of the catheter. In some embodiments, the wall thickness is substantially constant through the length of the catheter. In some embodiments, the wall thickness varies less than about 30%, 20%, 10%, or 5% over length of the catheter. In some embodiments, the proximal end of the interior catheter is configured to be pushable.

In some embodiments, a distal portion of the interior catheter is made of a soft material. In some embodiments, a distal portion of the interior catheter is made of a compliant material. In some embodiments, the interior catheter comprises a tip. In some embodiments, the tip comprises a soft and/or compliant material. In some embodiments, the tip is configured to be detachable. In some embodiments, the inner diameter of the interior catheter is configured to fit a guidewire. In some embodiments, the interior catheter is configured to deliver a composition to a target site. In some embodiments, the composition is an embolic material. In some embodiments, the interior catheter is configured to deliver a composition to a target site through a lumen of the interior catheter. In some embodiments, the composition comprises a particle, a coil, a liquid embolic, or any combination thereof. In some embodiments, the interior catheter is configured to be removed from the exterior catheter after delivering the composition. In some embodiments, a second interior catheter is configured to be placed in the exterior catheter after a first interior catheter is removed. In some embodiments, a third interior catheter is configured to be placed in the exterior catheter after the second interior catheter is removed. In some embodiments, the interior catheter may be removed and replaced by an unused interior catheter as many times as necessary.

Described herein are interior catheters comprising an outer diameter. In some embodiments, the outer diameter is sized to fit inside an inner diameter of an exterior catheter. In some embodiments, the outer diameter is sized to fit inside the inner diameter of the exterior catheter such that the interior and exterior catheters are configured to allow free movement between the interior and the exterior catheters. In some embodiments, the outer diameter of the interior catheter configured to fit inside the inner diameter of the exterior catheter such that a gap is created. In some embodiments, the gap is a difference between the outer diameter of the interior catheter and the inner diameter of the exterior catheter. In some embodiments, the gap is configured to allow free movement between the interior catheter and the exterior catheter. In some embodiments, the gap is about 0.0005 inches to about 0.004 inches. In some embodiments, about 0.0005 inches to about 0.004 inches, about 0.001 inches to about 0.0035 inches, about 0.0015 inches to about 0.003 inches, or about 0.002 inches to about 0.0025 inches. In some embodiments, the gap is about 0.0005 inches, about 0.001 inches, about 0.0015 inches, about 0.002 inches, about 0.0025 inches, about 0.003 inches, about 0.0035 inches, or about 0.004 inches. In some embodiments, the gap is at least about 0.0005 inches, about 0.001 inches, about 0.0015 inches, about 0.002 inches, about 0.0025 inches, about 0.003 inches, or about 0.0035 inches. In some embodiments, the gap is at most about 0.001 inches, about 0.0015 inches, about 0.002 inches, about 0.0025 inches, about 0.003 inches, about 0.0035 inches, or about 0.004 inches.

In some embodiments, the outer diameter of the interior catheter is substantially constant over the length of the catheter. In some embodiments, the outer diameter varies less than about 30%, 20%, 10%, or 5% over length of the catheter. In some embodiments, the outer diameter of the interior catheter is substantially constant, wherein the outer diameter of the interior catheter is configured to maximize an inner diameter of the interior catheter. In some embodiments, the outer diameter of the interior catheter is substantially constant, wherein the outer diameter of the interior catheter is configured to be compatible with the inner diameter of the exterior catheter. In some embodiments, the outer diameter of the interior catheter is substantially constant, wherein the outer diameter of the interior catheter is configured to maximize an inner diameter of the interior catheter and be compatible with the inner diameter of the exterior catheter. In some embodiments, the outer diameter of the interior catheter is configured to navigate to a distal blood vessel. In some embodiments, the outer diameter of the interior catheter is configured to navigate to a vessel of a Middle Meningeal Artery (MMA). In some embodiments, the outer diameter of the interior catheter is configured to navigate distally past a bifurcation of the MMA.

In some embodiments, the outer diameter comprises a proximal outer diameter and a distal outer diameter. In some embodiments, the proximal outer diameter is defined by measuring the diameter of the interior catheter at a proximal end of the interior catheter. In some embodiments, the distal outer diameter is defined by measuring the diameter of the interior catheter at a distal end of the interior catheter. In some embodiments, the proximal outer diameter of the interior catheter is greater than the distal outer diameter of the interior catheter. In some embodiments, the proximal outer diameter is greater than the distal outer diameter by about 0.0005 inches, about 0.00075 inches, about 0.001 inches, about 0.002 inches, about 0.0025 inches, about 0.003 inches, about 0.004 inches, about 0.005 inches, about 0.006 inches, about 0.007 inches, about 0.008 inches, about 0.009 inches, about 0.010 inches, about 0.011 inches, about 0.012 inches, about 0.0125 inches, about 0.013 inches, about 0.014 inches, about 0.015 inches, about 0.016 inches, about 0.017 inches, about 0.018 inches, about 0.019 inches, about 0.020 inches, about 0.021 inches, about 0.0225 inches, about 0.025 inches, about 0.0275 inches, or about 0.030 inches. In some embodiments, the proximal outer diameter decreases gradually.

In some embodiments, the outer diameter of the interior catheter is about 0.0075 inches, about 0.01 inches, about 0.0125 inches, about 0.015 inches, about 0.0175 inches, about 0.02 inches, about 0.0225 inches, about 0.025 inches, about 0.0275 inches, about 0.03 inches, about 0.0325 inches, about 0.035 inches, about 0.0375 inches, about 0.04 inches, about 0.0425 inches, about 0.045 inches, about 0.0475 inches, or about 0.05 inches. In some embodiments, the outer diameter of the interior catheter is about 0.012 inches to about 0.039 inches. In some embodiments, the outer diameter of the interior catheter is about 0.012 inches, about 0.015 inches, about 0.018 inches, about 0.021 inches, about 0.023 inches, about 0.025 inches, about 0.027 inches, about 0.029 inches, about 0.031 inches, about 0.033 inches, about 0.036 inches, or about 0.039 inches. In some embodiments, the outer diameter of the interior catheter is at least about 0.012 inches, about 0.015 inches, about 0.018 inches, about 0.021 inches, about 0.023 inches, about 0.025 inches, about 0.027 inches, about 0.029 inches, about 0.031 inches, about 0.033 inches, or about 0.036 inches. In some embodiments, the outer diameter of the interior catheter is at most about 0.015 inches, about 0.018 inches, about 0.021 inches, about 0.023 inches, about 0.025 inches, about 0.027 inches, about 0.029 inches, about 0.031 inches, about 0.033 inches, about 0.036 inches, or about 0.039 inches. In some embodiments, the outer diameter of the interior catheter is about 0.018 inches, about 0.019 inches, about 0.02 inches, about 0.021 inches, about 0.022 inches, about 0.023 inches, about 0.024 inches, about 0.025 inches, about 0.026 inches, about 0.027 inches, about 0.028 inches, or about 0.029 inches. In some embodiments, the outer diameter of the interior catheter is at least about 0.018 inches, about 0.019 inches, about 0.02 inches, about 0.021 inches, about 0.022 inches, about 0.023 inches, about 0.024 inches, about 0.025 inches, about 0.026 inches, about 0.027 inches, or about 0.028 inches. In some embodiments, the outer diameter of the interior catheter is at most about 0.019 inches, about 0.02 inches, about 0.021 inches, about 0.022 inches, about 0.023 inches, about 0.024 inches, about 0.025 inches, about 0.026 inches, about 0.027 inches, about 0.028 inches, or about 0.029 inches.

In some embodiments, the outer diameter comprises a proximal outer diameter. In some embodiments, the proximal outer diameter is measured at a proximal end of the catheter. In some embodiments, the outer diameter comprises a distal outer diameter. In some embodiments, the distal outer diameter is measured at a distal end of the catheter. In some embodiments, the outer diameter changes at most by about 25% along the length of the catheter from the proximal end to the distal end. In some embodiments, the outer diameter change is calculated by a difference of the proximal outer diameter and the distal outer diameter, divided by the distal outer diameter, multiplied by 100. In some embodiments, the outer diameter changes by at most about 0%, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, or at most about 25% along the length of the catheter. In some embodiments, the outer diameter changes by between about 0% and 10%, between about 1% and about 9%, between about 2% and about 8%, between about 3% and about 7%, between about 4% and about 6%, or about 5%. In some embodiments, the outer diameter changes by less than about 10%, less than about 9%, less than about 8%, less than about 7%, less than about 6%, less than about 5%, less than about 4%, less than about 3%, less than about 2%, less than about 1%, less than about 0% along the length of the catheter. In some embodiments, the outer diameter is substantially constant.

Described herein are interior catheters comprising an inner diameter. In some embodiments, the inner diameter of the interior catheter is configured to receive a guidewire. In some embodiments, the guidewire is configured to help the interior catheter navigate tortuous vasculature. In some embodiments, the guidewire is configured to help navigate the interior catheter to a first target site. In some embodiments, the guidewire is a 0.014 inch or a 0.018 inch guidewire. In some embodiments, the outer diameter of the interior catheter is configured to be substantially constant such that the inner diameter of the interior catheter is maximized.

In some embodiments, the inner diameter of the interior catheter is about 0.005 inches to about 0.025 inches. In some embodiments, the inner diameter of the interior catheter is about 0.005 inches, about 0.008 inches, about 0.01 inches, about 0.012 inches, about 0.014 inches, about 0.015 inches, about 0.016 inches, about 0.0165 inches, about 0.017 inches, about 0.018 inches, about 0.02 inches, or about 0.025 inches. In some embodiments, the inner diameter of the interior catheter is at least about 0.005 inches, about 0.008 inches, about 0.01 inches, about 0.012 inches, about 0.014 inches, about 0.015 inches, about 0.016 inches, about 0.0165 inches, about 0.017 inches, about 0.018 inches, or about 0.02 inches. In some embodiments, the inner diameter of the interior catheter is at most about 0.008 inches, about 0.01 inches, about 0.012 inches, about 0.014 inches, about 0.015 inches, about 0.016 inches, about 0.0165 inches, about 0.017 inches, about 0.018 inches, about 0.02 inches, or about 0.025 inches.

In some embodiments, the interior catheter comprises a wall thickness. In some embodiments, the wall thickness is measured as the difference between the outer diameter of the interior catheter and the inner diameter of the interior catheter. In some embodiments, the wall thickness of the interior catheter is substantially constant. In some embodiments, the wall thickness of the interior catheter is approximately equal to a wall thickness of an exterior catheter. In some embodiments, the wall thickness of the interior catheter is greater than the wall thickness of the exterior catheter. In some embodiments, the wall thickness of the interior catheter is less than the wall thickness of the exterior catheter. In some embodiments, the wall thickness of the interior catheter and the wall thickness of the exterior catheter are substantially constant.

In some embodiments, the wall thickness of the interior catheter is about 0.001 inches to about 0.023 inches. In some embodiments, the wall thickness of the interior catheter is about 0.001 inches, about 0.003 inches, about 0.005 inches, about 0.007 inches, about 0.009 inches, about 0.011 inches, about 0.013 inches, about 0.015 inches, about 0.017 inches, about 0.019 inches, about 0.021 inches, or about 0.023 inches. In some embodiments, the wall thickness of the interior catheter is at least about 0.001 inches, about 0.003 inches, about 0.005 inches, about 0.007 inches, about 0.009 inches, about 0.011 inches, about 0.013 inches, about 0.015 inches, about 0.017 inches, about 0.019 inches, or about 0.021 inches. In some embodiments, the wall thickness of the interior catheter is at most about 0.003 inches, about 0.005 inches, about 0.007 inches, about 0.009 inches, about 0.011 inches, about 0.013 inches, about 0.015 inches, about 0.017 inches, about 0.019 inches, about 0.021 inches, or about 0.023 inches.

In some embodiments, the interior catheter is soft. In some embodiments, the interior catheter is compliant. In some embodiments, the interior catheter is soft and compliant. In some embodiments, the interior catheter comprises an interior catheter body. In some embodiments, the interior catheter body comprises an interior tube. In some embodiments, in order to make a cut tube more flexible, a pitch (P) of a cut pattern may be decreased and/or a widths (W) of a bridges may be decreased. In some embodiments, the pitch (P) of the cut pattern is reduced to improve flexibility of an interior catheter body to allow for an interior catheter with sufficient flexibility and/or softness to traverse through torturous anatomy. In some embodiments, the width (W) of the bridge may be reduced to improve flexibility of an interior catheter body to allow for an interior catheter with enough flexibility to traverse through torturous anatomy. This may allow for the highest degree of flexibility and softness allowing the interior catheter to be advanced through more tortuous anatomy.

In some embodiments, the interior catheter comprises a tip. In some embodiments, the interior catheter tip comprises a detachable tip. In some embodiments, a distal portion of the interior catheter comprises the detachable tip. In some embodiments, the interior catheter comprises a detaching mechanism. In some embodiments, the detaching mechanism is configured to separate a connection between the interior catheter and the detachable tip. In some embodiments, the detachable tip is configured to reduce the risk of entrapment of the interior catheter in the vessel. In some embodiments, the detachable tip is configured to aid removal of the interior catheter from the vessel after a liquid embolic is delivered to a vessel. In some embodiments, the tip is made from a soft material. Non-limiting examples of the soft material include as a thermoplastic elastomer, silicone, polyurethane, or other biocompatible materials. In some embodiments, the tip is configured to be soft and compliant.

In some embodiments, the interior catheter comprises a proximal end. In some embodiments, the proximal end is configured to be pushable.

In some embodiments, the interior catheter is configured to be longer than an exterior catheter. In some embodiments, the interior catheter is configured to be a same or similar length of the exterior catheter. In some embodiments, the interior catheter is configured to be shorter than an exterior catheter.

In some embodiments, the length of the interior catheter is longer than the exterior catheter by about 0.5 cm to about 50 cm. In some embodiments, the length of the interior catheter is longer than the exterior catheter by about 0.5 cm to about 50 cm, about 1 cm to about 45 cm, about 5 cm to about 40 cm, about 10 cm to about 35 cm, about 15 cm to about 30 cm, about 20 cm to about 25 cm, or about 25 cm to about 30 cm. In some embodiments, the length of the interior catheter is longer than the exterior catheter by about 0.5 cm, about 1 cm, about 5 cm, about 10 cm, about 15 cm, about 20 cm, about 25 cm, about 30 cm, about 35 cm, about 40 cm, about 45 cm, or about 50 cm. In some embodiments, the length of the interior catheter is longer than the exterior catheter by at least about 0.5 cm, about 1 cm, about 5 cm, about 10 cm, about 15 cm, about 20 cm, about 25 cm, about 30 cm, about 35 cm, about 40 cm, or about 45 cm. In some embodiments, the length of the interior catheter is longer than the exterior catheter by at most about 1 cm, about 5 cm, about 10 cm, about 15 cm, about 20 cm, about 25 cm, about 30 cm, about 35 cm, about 40 cm, about 45 cm, or about 50 cm.

### Exterior Catheter

The catheter assembly and methods provided herein comprise a first catheter (e.g., interior catheter) and a second catheter (e.g., exterior catheter). In some embodiments, the exterior catheter comprises a laser cut tube (LCT). In some embodiments, the LCT comprises a first section comprising a plurality of first cuts. In some embodiments, the LCT comprises a second section adjacent to the first section, the second section comprising a plurality of second cut. In some embodiments, the LCT comprises a cut filler comprising a polymer that is filling at least one of a first cut of the plurality of the first cut and a second cut of the plurality of second cuts. In some embodiments, an interior catheter is configured to fit inside the exterior catheter. In some embodiments, the exterior catheter is configured to navigate through tortuous vessels. In some embodiments, the exterior catheter is configured to navigate through tortuous neurovasculature. In some embodiments, the exterior catheter is configured to navigate through a middle meningeal artery (MMA). In some embodiments, the exterior catheter is configured to navigate at least past a bifurcation of the MMA. In some embodiments, the exterior catheter is configured to navigate through a distal branch of the MMA. In some embodiments, the exterior catheter is configured to navigate through the MMA without wedging along the MMA before reaching a target site. In some embodiments, the exterior catheter comprises an inner diameter, an outer diameter, and a wall thickness. In some embodiments, the outer diameter comprises a proximal outer diameter. In some embodiments, the proximal outer diameter is defined by measuring the diameter of the exterior catheter at a proximal end of the exterior catheter. In some embodiments, the outer diameter comprises a distal outer diameter. In some embodiments, the distal outer diameter is defined by measuring the diameter of the exterior catheter at a distal end of the exterior catheter. In some embodiments, a ratio of the proximal outer diameter to the distal outer diameter is less than about 1.5. In some embodiments, the proximal outer diameter and the distal outer diameter are substantially equal. In some embodiments, the outer diameter is substantially constant. In some embodiments, the inner diameter is substantially constant. In some embodiments, the wall thickness is substantially constant. In some embodiments, the proximal end of the exterior catheter is configured to be pushable. In some embodiments, the exterior catheter is made of a soft material. In some embodiments, the exterior catheter is made of a compliant material. In some embodiments, the exterior catheter is made of a soft and a compliant material. In some embodiments, the exterior catheter comprises a tip. In some embodiments, the tip comprises a soft and/or compliant material. In some embodiments, the tip is configured to be detachable. In some embodiments, the exterior catheter is configured to deliver a composition to a target site. In some embodiments, the composition is an embolic material. In some embodiments, the exterior catheter is configured to deliver a composition to a target site through a lumen of the interior catheter. In some embodiments, the composition comprises a particle, a coil, a liquid embolic, or any combination thereof. In some embodiments, the interior catheter is configured to be removed from the exterior catheter after delivering an embolic. In some embodiments, a second interior catheter is configured to be placed in the exterior catheter after a first interior catheter is removed. In some embodiments, a third interior catheter is configured to be placed in the exterior catheter after the second interior catheter is removed. In some embodiments, the interior catheter may be removed and replaced by an unused interior catheter as many times as necessary.

Described herein are exterior catheters comprising an outer diameter. In some embodiments, the outer diameter is configured to allow the exterior catheter to reach a distal location in tortuous vascular anatomy. In some embodiments, the outer diameter is configured to allow the exterior catheter to reach a location where an embolization treatment may be performed. In some embodiments, the embolization treatment may comprise a Middle Meningeal Artery (MMA) embolization. In some embodiments, the location where an embolization treatment may be performed at a bifurcation of the MMA. In some embodiments, the location where an embolization treatment may be performed at a trunk of the MMA. In some embodiments, the outer diameter of the exterior catheter is configured to allow the exterior catheter to navigate at least to the bifurcation of the MMA. In some embodiments, the outer diameter of the exterior catheter is configured to allow the exterior catheter to navigate distally past the bifurcation of the MMA. In some embodiments, the outer diameter of the exterior catheter is configured to allow the exterior catheter to navigate at least past the bifurcation of the MMA to a branch of the MMA. In some embodiments, the outer catheter should be sized so as not to wedge along the MMA before reaching a target site.

In some embodiments, the outer diameter comprises a proximal outer diameter and a distal outer diameter. In some embodiments, the proximal outer diameter is defined by measuring the diameter of the exterior catheter at a proximal end of the exterior catheter. In some embodiments, the distal outer diameter is defined by measuring the diameter of the exterior catheter at a distal end of the exterior catheter. In some embodiments, the proximal outer diameter of the exterior catheter is greater than the distal outer diameter of the exterior catheter. In some embodiments, the proximal outer diameter is greater than the distal outer diameter by about 0.0005 inches, about 0.00075 inches, about 0.001 inches, about 0.002 inches, about 0.0025 inches, about 0.003 inches, about 0.004 inches, about 0.005 inches, about 0.006 inches, about 0.007 inches, about 0.008 inches, about 0.009 inches, about 0.010 inches, about 0.011 inches, about 0.012 inches, about 0.0125 inches, about 0.013 inches, about 0.014 inches, about 0.015 inches, about 0.016 inches, about 0.017 inches, about 0.018 inches, about 0.019 inches, about 0.020 inches, about 0.021 inches, about 0.0225 inches, about 0.025 inches, about 0.0275 inches, or about 0.030 inches. In some embodiments, the proximal outer diameter decreases gradually.

In some embodiments, the outer diameter of the exterior catheter is about 0.015 inches to about 0.06 inches. In some embodiments, the outer diameter of the exterior catheter is about 0.015 inches to about 0.06 inches, about 0.02 inches to about 0.05 inches, about 0.024 inches to about 0.045 inches, about 0.025 inches to about 0.04 inches, about 0.028 inches to about 0.037 inches, or about 0.031 inches to about 0.034 inches. In some embodiments, the outer diameter of the exterior catheter is about 0.015 inches, about 0.02 inches, about 0.024 inches, about 0.025 inches, about 0.028 inches, about 0.031 inches, about 0.034 inches, about 0.037 inches, about 0.04 inches, about 0.045 inches, about 0.05 inches, or about 0.06 inches. In some embodiments, the outer diameter of the exterior catheter is at least about 0.015 inches, about 0.02 inches, about 0.024 inches, about 0.025 inches, about 0.028 inches, about 0.031 inches, about 0.034 inches, about 0.037 inches, about 0.04 inches, about 0.045 inches, or about 0.05 inches. In some embodiments, the outer diameter of the exterior catheter is at most about 0.02 inches, about 0.024 inches, about 0.025 inches, about 0.028 inches, about 0.031 inches, about 0.034 inches, about 0.037 inches, about 0.04 inches, about 0.045 inches, about 0.05 inches, or about 0.06 inches.

In some embodiments, the outer diameter comprises a proximal outer diameter. In some embodiments, the proximal outer diameter is measured at a proximal end of the exterior catheter. In some embodiments, the outer diameter comprises a distal outer diameter. In some embodiments, the distal outer diameter is measured at a distal end of the exterior catheter. In some embodiments, the outer diameter changes at most by about 25%. In some embodiments, the outer diameter change is calculated by a difference of the proximal outer diameter and the distal outer diameter, divided by the distal outer diameter, multiplied by 100. In some embodiments, the outer diameter changes by about 0.1% to about 7.6%. In some embodiments, the outer diameter changes by about 0.1% to about 0.25%, about 0.1% to about 0.5%, about 0.1% to about 0.75%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 7.6%, about 0.25% to about 0.5%, about 0.25% to about 0.75%, about 0.25% to about 1%, about 0.25% to about 2%, about 0.25% to about 3%, about 0.25% to about 4%, about 0.25% to about 5%, about 0.25% to about 6%, about 0.25% to about 7%, about 0.25% to about 7.6%, about 0.5% to about 0.75%, about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 7.6%, about 0.75% to about 1%, about 0.75% to about 2%, about 0.75% to about 3%, about 0.75% to about 4%, about 0.75% to about 5%, about 0.75% to about 6%, about 0.75% to about 7%, about 0.75% to about 7.6%, about 1% to about 2%, about 1% to about 3%, about 1% to about 4%, about 1% to about 5%, about 1% to about 6%, about 1% to about 7%, about 1% to about 7.6%, about 2% to about 3%, about 2% to about 4%, about 2% to about 5%, about 2% to about 6%, about 2% to about 7%, about 2% to about 7.6%, about 3% to about 4%, about 3% to about 5%, about 3% to about 6%, about 3% to about 7%, about 3% to about 7.6%, about 4% to about 5%, about 4% to about 6%, about 4% to about 7%, about 4% to about 7.6%, about 5% to about 6%, about 5% to about 7%, about 5% to about 7.6%, about 6% to about 7%, about 6% to about 7.6%, or about 7% to about 7.6%. In some embodiments, the outer diameter changes by about 0.1%, about 0.25%, about 0.5%, about 0.75%, about 1%, about 2%, about 3%, about 4%, about 5%, about 6%, about 7%, or about 7.6%. In some embodiments, the outer diameter changes by at least about 0.1%, about 0.25%, about 0.5%, about 0.75%, about 1%, about 2%, about 3%, about 4%, about 5%, about 6%, or about 7%. In some embodiments, the outer diameter changes by at most about 0.25%, about 0.5%, about 0.75%, about 1%, about 2%, about 3%, about 4%, about 5%, about 6%, about 7%, or about 7.6%. In some embodiments, the outer diameter is substantially constant.

Described herein are exterior catheters comprising an inner diameter. In some embodiments, the inner diameter of the exterior catheter is configured to fit an outer diameter of an interior catheter. In some embodiments, the inner diameter of the exterior catheter is configured to fit an outer diameter of an interior catheter such that a gap is created. In some embodiments, the gap is a difference between the outer diameter of the interior catheter and the inner diameter of the exterior catheter. In some embodiments, the gap is configured to allow free movement between the interior catheter and the exterior catheter. In some embodiments, the gap is about 0.0005 inches to about 0.004 inches. In some embodiments, about 0.0005 inches to about 0.004 inches, about 0.001 inches to about 0.0035 inches, about 0.0015 inches to about 0.003 inches, or about 0.002 inches to about 0.0025 inches. In some embodiments, the gap is about 0.0005 inches, about 0.001 inches, about 0.0015 inches, about 0.002 inches, about 0.0025 inches, about 0.003 inches, about 0.0035 inches, or about 0.004 inches. In some embodiments, the gap is at least about 0.0005 inches, about 0.001 inches, about 0.0015 inches, about 0.002 inches, about 0.0025 inches, about 0.003 inches, or about 0.0035 inches. In some embodiments, the gap is at most about 0.001 inches, about 0.0015 inches, about 0.002 inches, about 0.0025 inches, about 0.003 inches, about 0.0035 inches, or about 0.004 inches.

In some embodiments, the outer diameter of the interior catheter is substantially constant over the length of the catheter. In some embodiments, the outer diameter of the interior catheter is substantially constant, wherein the outer diameter of the interior catheter is configured to maximize an inner diameter of the interior catheter. In some embodiments, the outer diameter of the interior catheter is substantially constant, wherein the outer diameter of the interior catheter is configured to be compatible with the inner diameter of the exterior catheter. In some embodiments, the outer diameter of the interior catheter is substantially constant, wherein the outer diameter of the interior catheter is configured to maximize an inner diameter of the interior catheter and be compatible with the inner diameter of the exterior catheter. In some embodiments, the inner diameter of the exterior catheter is configured to fit a guidewire.

In some embodiments, the exterior catheter comprises a wall thickness. In some embodiments, the wall thickness is measured as the difference between the outer diameter of the interior catheter and the inner diameter of the interior catheter. In some embodiments, the wall thickness of the exterior catheter is substantially constant. In some embodiments, the wall thickness of the exterior catheter is approximately equal to a wall thickness of an interior catheter. In some embodiments, the wall thickness of the interior catheter is greater than the wall thickness of the exterior catheter. In some embodiments, the wall thickness of the interior catheter is less than the wall thickness of the exterior catheter. In some embodiments, the wall thickness of the interior catheter and the wall thickness of the exterior catheter are substantially constant.

In some embodiments, the wall thickness of the exterior catheter is about 0.001 inches to about 0.023 inches. In some embodiments, the wall thickness of the exterior catheter is about 0.001 inches to about 0.003 inches, about 0.001 inches to about 0.005 inches, about 0.001 inches to about 0.007 inches, about 0.001 inches to about 0.009 inches, about 0.001 inches to about 0.011 inches, about 0.001 inches to about 0.013 inches, about 0.001 inches to about 0.015 inches, about 0.001 inches to about 0.017 inches, about 0.001 inches to about 0.019 inches, about 0.001 inches to about 0.021 inches, about 0.001 inches to about 0.023 inches, about 0.003 inches to about 0.005 inches, about 0.003 inches to about 0.007 inches, about 0.003 inches to about 0.009 inches, about 0.003 inches to about 0.011 inches, about 0.003 inches to about 0.013 inches, about 0.003 inches to about 0.015 inches, about 0.003 inches to about 0.017 inches, about 0.003 inches to about 0.019 inches, about 0.003 inches to about 0.021 inches, about 0.003 inches to about 0.023 inches, about 0.005 inches to about 0.007 inches, about 0.005 inches to about 0.009 inches, about 0.005 inches to about 0.011 inches, about 0.005 inches to about 0.013 inches, about 0.005 inches to about 0.015 inches, about 0.005 inches to about 0.017 inches, about 0.005 inches to about 0.019 inches, about 0.005 inches to about 0.021 inches, about 0.005 inches to about 0.023 inches, about 0.007 inches to about 0.009 inches, about 0.007 inches to about 0.011 inches, about 0.007 inches to about 0.013 inches, about 0.007 inches to about 0.015 inches, about 0.007 inches to about 0.017 inches, about 0.007 inches to about 0.019 inches, about 0.007 inches to about 0.021 inches, about 0.007 inches to about 0.023 inches, about 0.009 inches to about 0.011 inches, about 0.009 inches to about 0.013 inches, about 0.009 inches to about 0.015 inches, about 0.009 inches to about 0.017 inches, about 0.009 inches to about 0.019 inches, about 0.009 inches to about 0.021 inches, about 0.009 inches to about 0.023 inches, about 0.011 inches to about 0.013 inches, about 0.011 inches to about 0.015 inches, about 0.011 inches to about 0.017 inches, about 0.011 inches to about 0.019 inches, about 0.011 inches to about 0.021 inches, about 0.011 inches to about 0.023 inches, about 0.013 inches to about 0.015 inches, about 0.013 inches to about 0.017 inches, about 0.013 inches to about 0.019 inches, about 0.013 inches to about 0.021 inches, about 0.013 inches to about 0.023 inches, about 0.015 inches to about 0.017 inches, about 0.015 inches to about 0.019 inches, about 0.015 inches to about 0.021 inches, about 0.015 inches to about 0.023 inches, about 0.017 inches to about 0.019 inches, about 0.017 inches to about 0.021 inches, about 0.017 inches to about 0.023 inches, about 0.019 inches to about 0.021 inches, about 0.019 inches to about 0.023 inches, or about 0.021 inches to about 0.023 inches. In some embodiments, the wall thickness of the exterior catheter is about 0.001 inches, about 0.003 inches, about 0.005 inches, about 0.007 inches, about 0.009 inches, about 0.011 inches, about 0.013 inches, about 0.015 inches, about 0.017 inches, about 0.019 inches, about 0.021 inches, or about 0.023 inches. In some embodiments, the wall thickness of the exterior catheter is at least about 0.001 inches, about 0.003 inches, about 0.005 inches, about 0.007 inches, about 0.009 inches, about 0.011 inches, about 0.013 inches, about 0.015 inches, about 0.017 inches, about 0.019 inches, or about 0.021 inches. In some embodiments, the wall thickness of the exterior catheter is at most about 0.003 inches, about 0.005 inches, about 0.007 inches, about 0.009 inches, about 0.011 inches, about 0.013 inches, about 0.015 inches, about 0.017 inches, about 0.019 inches, about 0.021 inches, or about 0.023 inches.

In some embodiments, the exterior catheter is soft. In some embodiments, the exterior catheter is compliant. In some embodiments, the exterior catheter is soft and compliant. In some embodiments, the exterior catheter comprises an interior catheter body. In some embodiments, the exterior catheter body comprises an interior tube. In some embodiments, in order to make a cut tube more flexible, a pitch (P) of a cut pattern may be decreased and/or a widths (W) of a bridges may be decreased. In some embodiments, the pitch (P) of the cut pattern is reduced to improve flexibility of an exterior catheter body to allow for an exterior catheter with sufficient flexibility and/or softness to traverse through torturous anatomy. In some embodiments, the width (W) of the bridge may be reduced to improve flexibility of an exterior catheter body to allow for an exterior catheter with enough flexibility to traverse through torturous anatomy. This may allow for the highest degree of flexibility and softness allowing the interior catheter to be advanced through more tortuous anatomy.

In some embodiments, the exterior catheter comprises a tip. In some embodiments, the exterior catheter tip comprises a detachable tip. In some embodiments, a distal portion of the exterior catheter comprises the detachable tip. In some embodiments, the exterior catheter comprises a detaching mechanism. In some embodiments, the detaching mechanism is configured to separate a connection between the exterior catheter and the detachable tip. In some embodiments, the detachable tip is configured to reduce the risk of entrapment of the exterior catheter in the vessel. In some embodiments, the detachable tip is configured to aid removal of the exterior catheter from the vessel after a liquid embolic is delivered to a vessel.

In some embodiments, the exterior catheter comprises a proximal end. In some embodiments, the proximal end is configured to be pushable.

In some embodiments, the interior catheter is configured to be longer than an exterior catheter. In some embodiments, the interior catheter is configured to be a same or similar length of the exterior catheter. In some embodiments, the interior catheter is configured to be shorter than an exterior catheter.

In some embodiments, the length of the interior catheter is longer than the exterior catheter by about 0.5 cm to about 50 cm. In some embodiments, the length of the interior catheter is longer than the exterior catheter by about 0.5 cm to about 50 cm, about 1 cm to about 45 cm, about 5 cm to about 40 cm, about 10 cm to about 35 cm, about 15 cm to about 30 cm, about 20 cm to about 25 cm, or about 25 cm to about 30 cm. In some embodiments, the length of the interior catheter is longer than the exterior catheter by about 0.5 cm, about 1 cm, about 5 cm, about 10 cm, about 15 cm, about 20 cm, about 25 cm, about 30 cm, about 35 cm, about 40 cm, about 45 cm, or about 50 cm. In some embodiments, the length of the interior catheter is longer than the exterior catheter by at least about 0.5 cm, about 1 cm, about 5 cm, about 10 cm, about 15 cm, about 20 cm, about 25 cm, about 30 cm, about 35 cm, about 40 cm, or about 45 cm. In some embodiments, the length of the interior catheter is longer than the exterior catheter by at most about 1 cm, about 5 cm, about 10 cm, about 15 cm, about 20 cm, about 25 cm, about 30 cm, about 35 cm, about 40 cm, about 45 cm, or about 50 cm.

### Compositions for Delivery

Described herein are catheter systems configured to deliver a therapeutic. In some embodiments, the therapeutic is an embolic. In some embodiments, the embolic is configured to obstruct blood flow of a target vessel. In some embodiments, the embolic is configured to stop blood flow of the target vessel. In some embodiments, the embolic is configured to reduce bleeding from a target vessel. In some embodiments, the embolic is configured to eliminate bleeding from a target vessel. In some embodiments, the embolic is configured to reduce blood pressure of a target vessel. In some embodiments, the embolic is configured to eliminate blood pressure of a target vessel. In some embodiments, the interior catheter is configured to deliver an embolic to a target vessel to treat a blood vessel abnormality. In some embodiments, the blood vessel abnormality is a subdural hematoma (SDH). In some embodiments, the subdural hematoma (SDH) is chronic. In some embodiments, a SDH may cause compression of a brain of an individual. In some embodiments, the blood vessel abnormality is an arteriovenous malformation (AVM). In some embodiments, the blood vessel abnormality is an aneurysm. In some embodiments, a Middle Meningeal Artery (MMA) comprises the target vessel. In some embodiments, the interior catheter delivers an embolic into the MMA. In some embodiments, the interior catheter delivers an embolic into the MMA such that the blood flow is obstructed. In some embodiments, the interior catheter is configured to deliver a liquid embolic to a first target site, wherein the first target site is located in the MMA, wherein the liquid embolic is configured to treat a SDH.

In some embodiments, the catheter system is configured to deliver an embolic to a target site. In some embodiments, the catheter system is configured to deliver a liquid embolic to a target site. In some embodiments, the catheter system is configured to deliver a coil to a target site. In some embodiments, the catheter system is configured to deliver a particle embolic to a target site. In some embodiments, the embolic comprises a liquid embolic, a coil, a particle embolic, or any combination thereof. In some embodiments, the liquid embolic comprises a polymer. In some embodiments, the polymer comprises a polyethylene glycol. In some embodiments, the polymer comprises an acrylate. In some embodiments, the polymer comprises a cyanoacrylate derivative. In some embodiments, the polymer comprises N-butyl-2-cyanoacrylate (NBCA). In some embodiments, the polymer comprises a copolymer. In some embodiments, the copolymer comprises ethylene vinyl alcohol. In some embodiments, the liquid embolic comprises the copolymer and a solvent. In some embodiments, the copolymer is dissolved in a solvent. In some embodiments, the solvent is dimethyl sulfoxide (DMSO). In some embodiments, the solvent is N-methyl pyrrolidone (NMP). In some embodiments, the embolic comprises a radiopacifying agent. In some embodiments, the radiopacifying agent comprises tantalum, iodine, or any combination thereof. In some embodiments, the liquid embolic comprises a hydrophobic injectable liquid. In some embodiments, the hydrophobic injectable liquid is a precipitating hydrophobic injectable liquid. In some embodiments, the liquid embolic comprises a polyvinyl alcohol. In some embodiments, the liquid embolic comprises an alginate. In some embodiments, the liquid embolic comprises a shear-thinning biomaterial.

In some embodiments, the embolic comprises a mechanism of vessel occlusion. In some embodiments, the vessel occlusion may be temporary. In some embodiments, the vessel occlusion may be permanent. In some embodiments, the mechanism of vessel occlusion comprises a polymerization reaction, wherein the embolic is configured to polymerize upon exposure to an initiator. In some embodiments, the polymerization reaction of the embolic is configured to form an occlusive cast in the blood vessel upon exposure to an initiator. In some embodiments, the initiator are ions. In some embodiments, the ions are found in the blood. In some embodiments, the polymerization reaction is configured to have a controlled rate of polymerization. In some embodiments, the rate of polymerization may be modified by a polymerization retardant. In some embodiments, the mechanism of vessel occlusion comprises a precipitation reaction, wherein a polymer is configured to precipitate out of a solution upon exposure to a precipitation stimulant. In some embodiments, the precipitation reaction is configured to form an occlusive cast in the blood vessel. In some embodiments, the occlusive cast in the blood vessel formed by the precipitation reaction is a gel. In some embodiments, the mechanism of vessel occlusion comprises a phase transition. In some embodiments, the phase transition of the embolic is configured to occur under specified conditions. Non-limiting examples of conditions include temperature, pH, ionic concentrations, or electric fields. In some embodiments, a polymer is configured to undergo the phase transition.

In some embodiments, the embolic comprises a coil. In some embodiments, the coil is configured to have thrombogenic properties to block blood flow. In some embodiments, the embolic comprises particles. In some embodiments, the particles comprise polyvinyl alcohol (PVA) particles. In some embodiments, the PVA particles are non-spherical. In some embodiments, the PVA particles are spherical. In some embodiments, the PVA particles are calibrated. In some embodiments, the PVA particles are non-calibrated. In some embodiments, the particles are configured to adhere to a vessel wall. In some embodiments, the particles are configured to form an intravascular lattice. In some embodiments, the intravascular lattice is configured to occlude the vessel. In some embodiments, the particles are configured to induce an inflammatory reaction. In some embodiments, the particles are configured to induce an inflammatory reaction to cause vessel necrosis.

### Methods of Use

Provided herein are methods for delivering a therapeutic to a target site in an subject. In some embodiments, the method comprises: positioning an access catheter into a vessel in an individual at a first site, advancing an outer catheter through the access catheter and into the vessel to a position that is proximal to a target site, advancing an interior catheter through the exterior catheter and into the vessel to a target site, and delivering a composition through a lumen of the interior catheter to the target site, wherein the composition is configured to prevent a leakage of blood from the vessel.

In some embodiments, the method comprises: positioning an access catheter into a vessel in an individual at a first site, advancing an outer catheter through the access catheter and into the vessel to a position that is proximal to a target site, advancing an interior catheter through the exterior catheter and into the vessel to a target site, and delivering a composition through a lumen of the interior catheter to the target site, wherein the composition is configured to prevent a leakage of blood from the vessel, removing the interior catheter from the exterior catheter, advancing the exterior catheter to a second target site, and delivering a composition through a lumen of the second interior catheter to the second target site.

In some embodiments, the method comprises: positioning an access catheter into a vessel in an individual at a first site, advancing an outer catheter through the access catheter and into the vessel to a position that is proximal to a target site, advancing an interior catheter through the exterior catheter and into the vessel to a target site, and delivering a composition through a lumen of the interior catheter to the target site, wherein the composition is configured to prevent a leakage of blood from the vessel removing the interior catheter from the exterior catheter, advancing a second interior catheter through the exterior catheter to a second target site, and delivering a composition through a lumen of the second interior catheter to the second target site. In some embodiments, the catheter assembly is configured to preserve a position proximal to the one or more target sites. In these instances, the preserved position of the exterior catheter may help to for reduce the time required to reach the target site(s) and/or provide a faster treatment time.

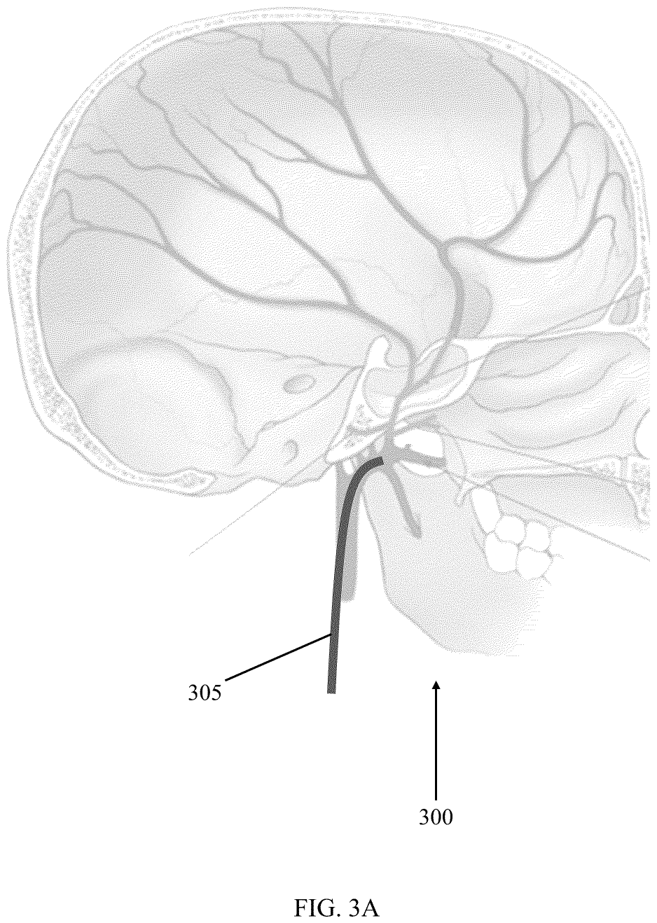

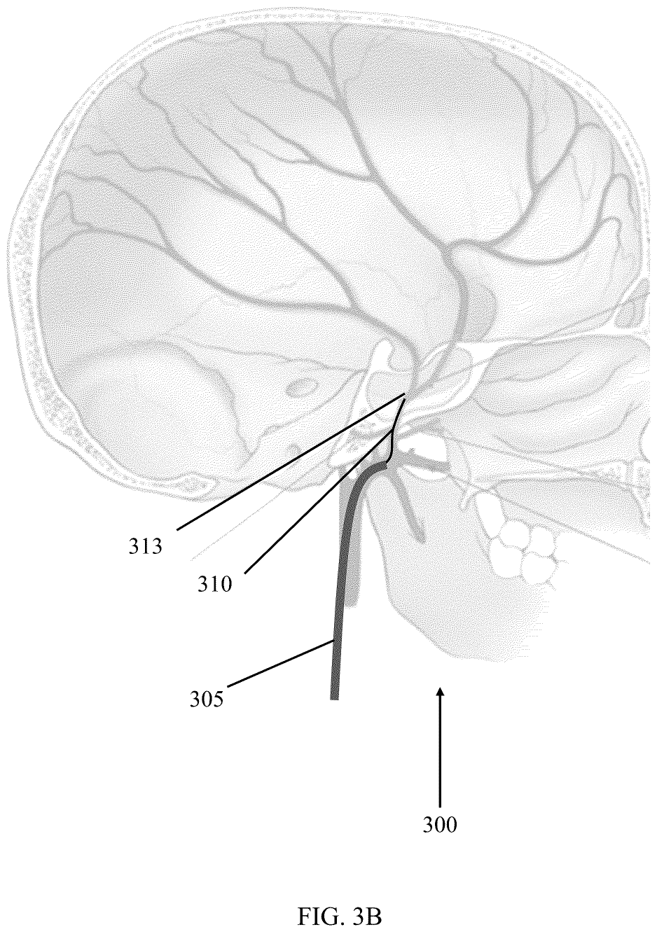

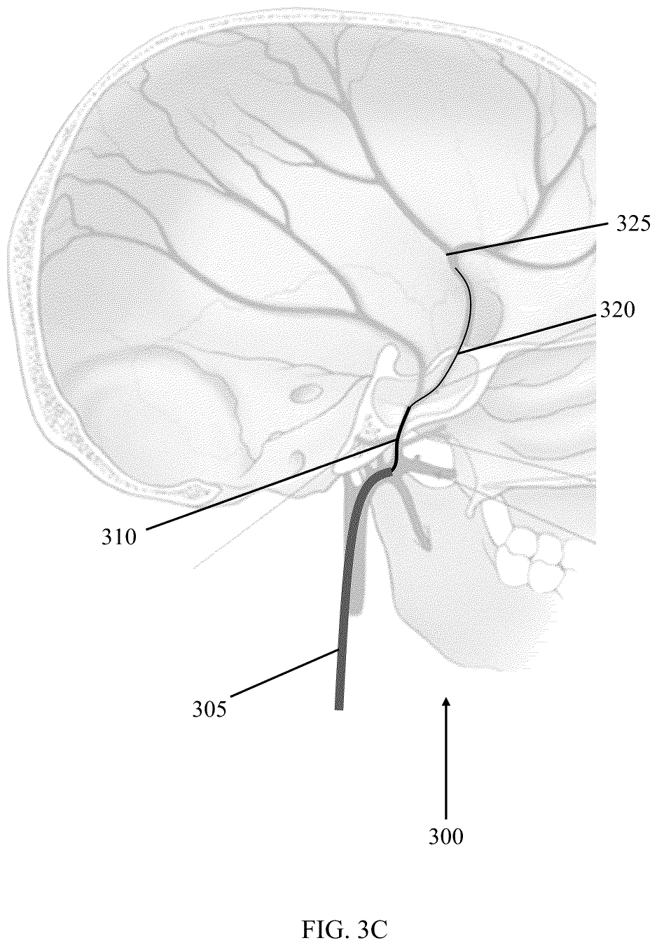

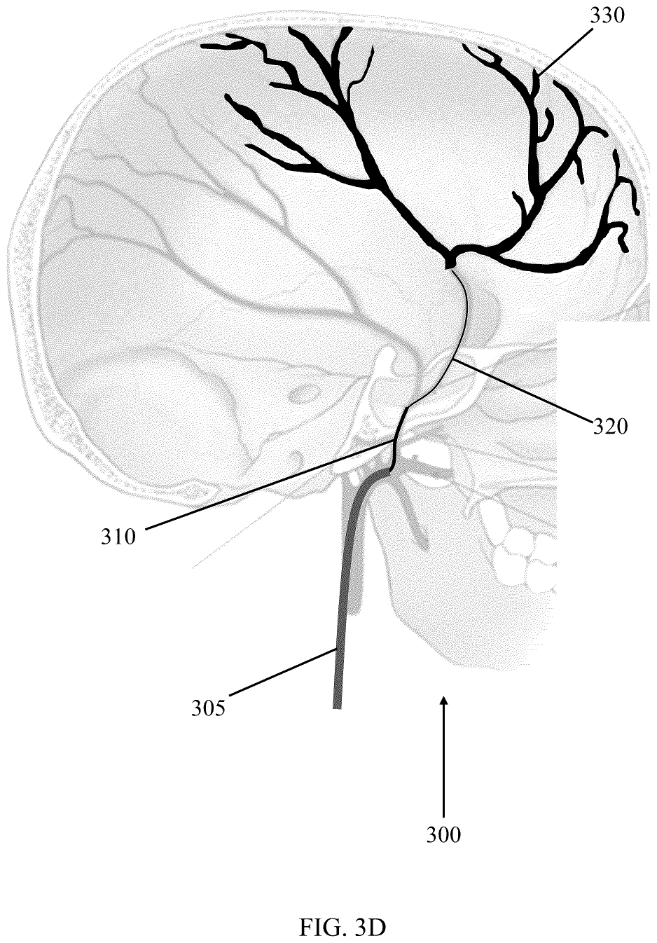

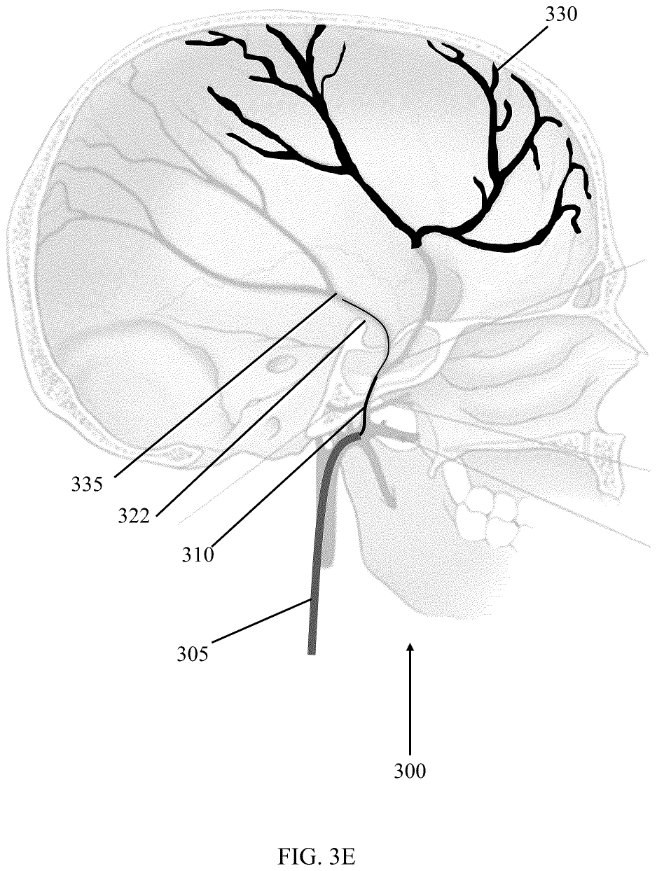

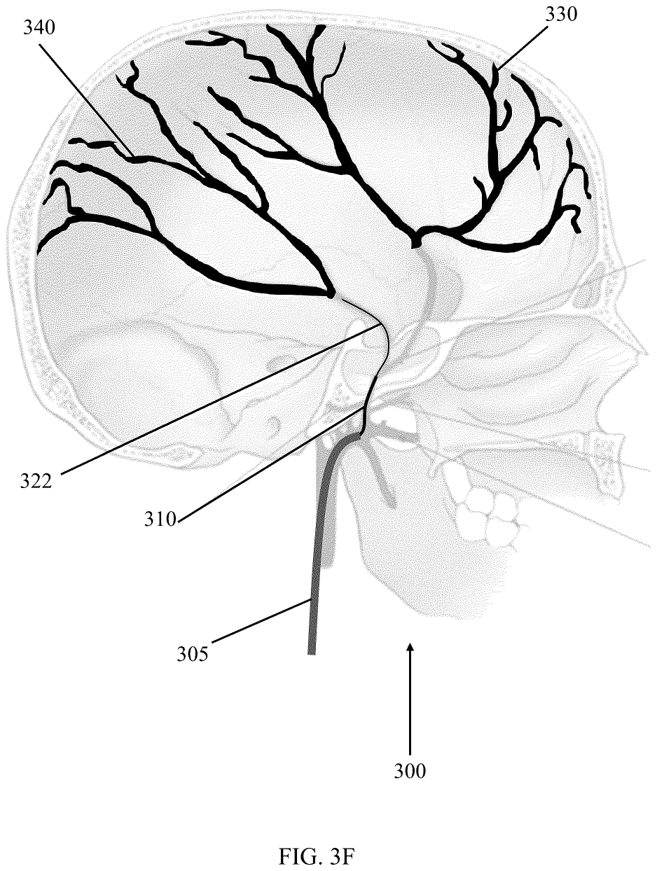

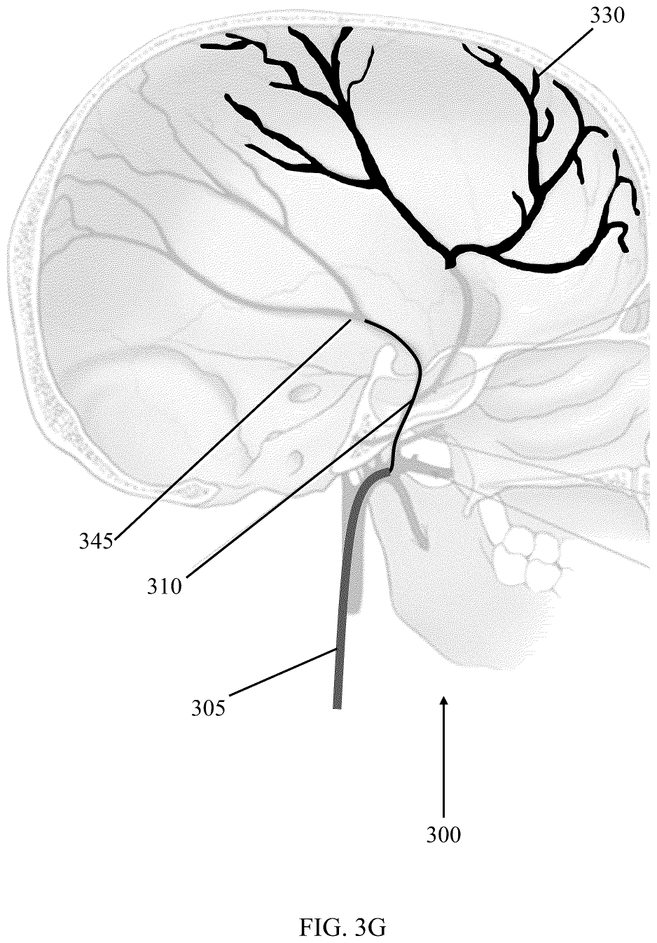

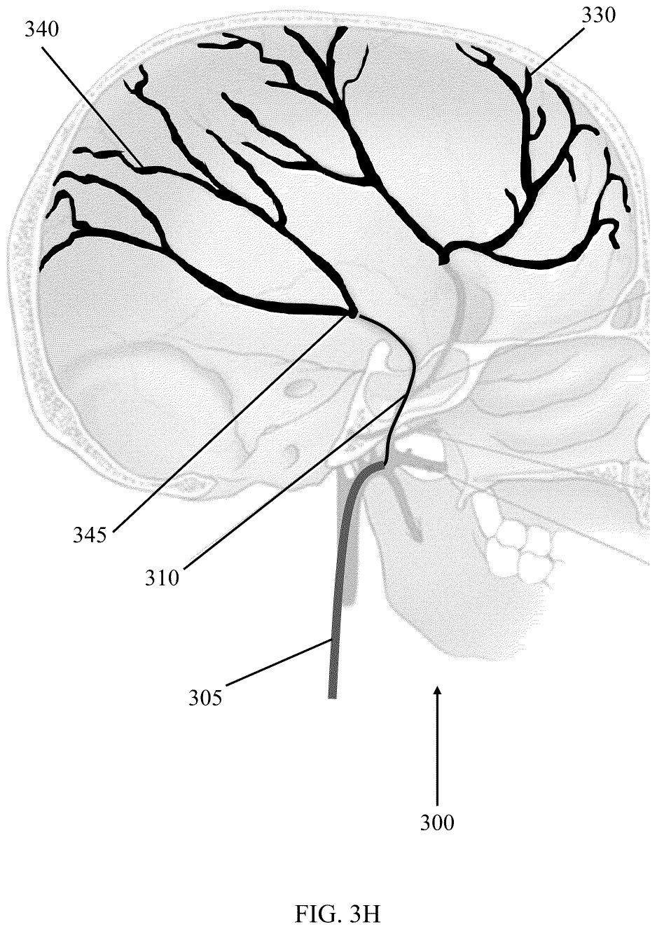

FIGS. 3A-3I illustrate progressions of using the devices and methods disclosed herein to deliver a therapeutic to a target site. FIG. 3A depicts a diagram of a subject 300 undergoing a middle meningeal (MMA) embolization. An access catheter 305 is advanced through vessels of a subject. The access catheter 305 is navigated through the vessels to a first site. In some embodiments, the first site is near the origin of the MMA, where the MMA forms from an internal maxillary artery (IMAX). In some embodiments, the access catheter is not advanced past (i.e., distally) the origin of the MMA. As seen in FIG. 3B, an exterior catheter 310 is advanced through the access catheter 305. The exterior catheter may advance past a distal end of the access catheter 305; i.e., the exterior catheter may advance past the origin of the MMA. The exterior catheter 310 may advance through the MMA to a position near a bifurcation 313 of the MMA. In some embodiments, the exterior catheter 310 may advance through the MMA to a position proximal to a first target site. As seen in FIG. 3C, an interior catheter 320 may advance through the exterior catheter 310, through the MMA to a first target site 325. The first target site 325 may be a branch of the MMA. In some embodiments, the first target site 325 is a distal branch of the MMA. A guidewire may be positioned in the interior catheter. After the interior catheter is advanced to the first target site 325, a composition may be delivered through a lumen of the interior catheter 320. The composition may comprise an embolic. In some embodiments, the composition comprises a liquid embolic. After delivery, the liquid embolic may form a cast 330 in the shape of the target vessel(s), as shown by FIG. 3D. In some embodiments, the cast 330 is configured to prevent a leakage of blood from the vessel. After delivering a composition, the interior catheter 320 may be retracted from the target site and the interior catheter 320 may be removed from the exterior catheter 310. A lumen of the exterior catheter 310 is preserved and free of liquid embolic. A guidewire may be positioned in the lumen of the exterior catheter without exposing the guidewire to the liquid embolic, which could otherwise render the guidewire unusable.

After removal of the interior catheter, a second interior catheter 322 may advance through the exterior catheter 310, through the MMA to a second target site 335, as seen in FIG. 3E. In some embodiments, an operator may use the second interior catheter 322 because of the operator's preference. In some embodiments, the operator may use the second interior catheter 322 because of one of more technical reasons. A non-limiting example of a technical reason is that the outer diameter of the exterior catheter is too large to reach the second target site 335. The second target site 335 may be a branch of the MMA. In some embodiments, the second target site 335 is a distal branch of the MMA. After the second interior catheter 322 is advanced to the second target site 335, a composition may be delivered through a lumen of the second interior catheter 322. The composition may comprise an embolic. In some embodiments, the composition comprises a liquid embolic. After delivery, the liquid embolic may form a second cast 340 in the shape of the target vessel(s), as shown by FIG. 3F. After delivering a composition, the second interior catheter 322 may be retracted from the target site and the second interior catheter 322 may be removed from the exterior catheter 310. After removal of the second interior catheter 322, additional interior catheters may be introduced in a similar fashion as demonstrated by FIGS. 3E-3F. FIG. 3I shows the resulting casts 330 and 340 after being treated with the composition. The casts are configured to prevent bleeding from the target vessels.

After removal of the interior catheter, the exterior catheter 310 may be advanced to a second target site 345, as shown in FIG. 3G. Any tortuosity that the exterior catheter had navigated up to its first location 313 does not need to be re-navigated, saving time compared to if a single catheter assembly was used. In those cases, the single catheter would be filled with liquid embolic and need to be removed from the vessel. A new catheter would then be advanced through the access catheter to the second target site, thus having to re-navigate tortuous anatomy between the distal end of the access catheter and the first location 313. The second target site 345 may be a branch of the MMA. In some embodiments, the second target site 345 is a distal branch of the MMA. After the exterior catheter 310 is advanced to the second target site 345, a composition may be delivered through a lumen of the exterior catheter 310. The composition may comprise an embolic. In some embodiments, the composition comprises a liquid embolic. After delivery, the liquid embolic may form a second cast 340 in the shape of the target vessel(s), as shown by FIG. 3H. After delivering a composition, the exterior catheter 310 may be retracted from the target site and may be removed from the vessel. The access catheter 305 may also be removed from the vessel. FIG. 3I shows the resulting casts 330 and 340 after being treated with a composition. The casts are configured to prevent bleeding from the target vessels. In some embodiments, the composition provided herein is used.

In some embodiments, the first target site is a blood vessel malformation. In some embodiments, the blood vessel abnormality is a subdural hematoma (SDH). In some embodiments, the subdural hematoma (SDH) is chronic. In some embodiments, the blood vessel abnormality is an arteriovenous malformation (AVM). In some embodiments, the blood vessel abnormality is an aneurysm. In some embodiments, a Middle Meningeal Artery (MMA) comprises the target vessel.

In some embodiments, the first site comprises an internal maxillary artery (IMAX). In some embodiments, the first site comprises an origin of the MMA from the IMAX. In some embodiments, an access catheter is positioned at the IMAX. In some embodiments, the access catheter is positioned at the origin of the MMA. In some embodiments, the access catheter is not advanced past (i.e., distally) the origin of the MMA.

### Laser Cut Catheter (LCT)

Described herein are catheters having variable flexibility and/or stiffness along its longitudinal length and methods of preparing such catheters. In some embodiments, the catheter comprises a catheter body comprising a first section comprising a plurality of first cuts; a second section adjacent to the first section, the second section comprising a plurality of second cuts; and a cut filler comprising a polymer that is filling at least one of a first cut of the plurality of the first cut and a second cut of the plurality of second cuts. In some embodiments, the catheter further comprises a liner (also referred herein as inner liner) and an outer jacket (also referred herein as outer liner). In some embodiments, the outer jacket is in contact with the cut filler. In some embodiments, the outer jacket is bonded with the cut filler. In some embodiments, the plurality of first cuts and/or the plurality of second cuts are formed by a laser. In some embodiments, the first cut of the plurality of first cuts has an average first kerf that is wider than an average kerf of the second cut of the plurality of second cuts. In some embodiments, the first section is more flexible than the second section. In some embodiments, the second section has a higher stiffness than the first section. In some embodiments, the cut filler is difficult to handle (e.g., very soft) and may fill the cuts with an aid of an outer backing layer. In some embodiments, the outer backing layer improves handleability of the cut filler. In some embodiments, the outer backing layer allows for easier application of the cut filler than without the outer backing layer. In some embodiments, the outer jacket comprises the outer backing layer.

In some embodiments, the catheter body comprises a tube having a lumen therethrough. In some embodiments, the catheter body is configured to have a variable stiffness and/or flexibility across different portions longitudinally. In some embodiments, the catheter body provides mechanical support for the catheter. In some embodiments, the catheter body is configured to be relatively rigid along at least a portion of the catheter body. In some embodiments, the catheter body may be configured to be less rigid along at least a portion of the catheter body.

In some embodiments, the first section comprising the plurality of first cuts cut is stretched longitudinally to a first stretch ratio (e.g., ratio of first stretched distance over the unstretched distance). In some embodiments, the first stretch ratio is at least about 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio is at most about 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio ranges from about 1.01 to about 10, about 1.1 to about 5, or about 1.1 to about 2

In some embodiments, the second section comprising the plurality of second cuts cut is stretched longitudinally to a second stretch ratio (e.g., ratio of second stretched distance over the unstretched distance). In some embodiments, the first stretch ratio is greater than the second stretch ratio. In some embodiments, the first stretch ratio is the same as the second stretch ratio. In some embodiments, no stretch is applied to the second portion. In some embodiments, the first stretch ratio is at least about 1, 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio is at most about 1, 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio ranges from about 1 to about 10, about 1 to about 5, or about 1 to about 2. In some embodiments, the second stretch ratio is 1.

In some embodiments, the catheter comprises an outer jacket covering at least an outer surface of the catheter body. In some embodiments, the outer jacket comprises a polymer.

In some embodiments, the catheter comprises a liner covering at least a portion of a lumen of the catheter body. In some embodiments, the liner comprises a material having a low coefficient of friction. In some embodiments, the material is a polymer having a low coefficient of friction. Such material may help to advance one or more structures through the lumen of the catheter body. For example, the liner may comprise a polymer having a low coefficient of friction. In some embodiments, the low coefficient of friction allows a guide wire to be advanced through the central lumen with little to no resistance caused by contact between guidewire with the liner.

The catheter may be modified to have different configurations to allow for navigation through different internal tissue structures, including but not limited to arteries, veins, capillaries, organs, or tissue layers. In some embodiments, the lumen of the catheter is configured to allow the passage of a second catheter and/or a guidewire through it. In some embodiments, the second catheter and/or a guidewire may be introduced through the lumen of the catheter during a procedure. In some embodiments, the second catheter and/or a guidewire may be removed from the lumen of the catheter during a procedure. In some embodiments, one or more of the catheter bodies, the liner, and the outer jacket may be configured to achieve a desired flexibility and/or stiffness per unit of length along the catheter. In some embodiments, the second catheter may have a tapered tip. In some embodiments, the catheter provided herein may be delivered to the target site through a lumen of a sheath. In some embodiments, the catheter provided herein may fit within a lumen of a sheath.

In some embodiments, the catheter comprises a first section having a first stiffness. In some embodiments, the catheter comprises a second section having a second stiffness. In some embodiments, the second stiffness is greater than the first stiffness. In some embodiments, the first section is distal to the second section. In some embodiments, the first section is proximal to the second section. In some embodiments, the first stiffness is at least about 1%, 5%, 10%, 15%, 20%, 25%, 30%, 35%, 40%, 45%, 50%, 60%, 70%, 80%, 90%, or 100% less than the second stiffness. In some embodiments, the first stiffness is at most about 10%, 15%, 20%, 25%, 30%, 35%, 40%, 45%, 50%, 60%, 70%, 80%, 90%, 100%, 200%, 300%, 400%, 500%, 600%, 700%, 800%, 900%, or 1000% less than the second stiffness.

In some embodiments, the first section is more flexible than the second section. In some embodiments, the first flexibility is at least about 1%, 5%, 10%, 15%, 20%, 25%, 30%, 35%, 40%, 45%, 50%, 60%, 70%, 80%, 90%, or 100% greater than the second flexibility. In some embodiments, the first flexibility is at most about 10%, 15%, 20%, 25%, 30%, 35%, 40%, 45%, 50%, 60%, 70%, 80%, 90%, 100%, 200%, 300%, 400%, 500%, 600%, 700%, 800%, 900%, or 1000% greater than the second flexibility.

Described herein are methods of preparing a variable flexible catheter and such catheters. In some embodiments, a catheter comprises an inner liner, an outer jacket, and a catheter body. In some embodiments, the catheter body is positioned between the inner liner and the outer jacket. In some embodiments, the catheter body comprises a tube. In some embodiments, the catheter body comprises a plurality of cuts, thereby comprising a cut tube. In some embodiments, the plurality of cuts comprise a first plurality of cuts and a second plurality of cuts.

In some embodiments, the catheter body comprises a cut tube. The cut tube may comprise one or more cuts forming a cut pattern. The cut tube may comprise a plurality of interrupted cuts forming an interrupted spiral cut pattern along at least a portion of the tube. In some embodiments, the one or more cuts may be in the longitudinal direction, the axial direction, or a combination of thereof. In some embodiments, the shape of the cuts may be rectangular, circular, oval, oblong, elliptical, s-shaped, or the like. The cuts may be formed into any shape configured to alter one or more features of the tube or a catheter comprising the tube. In some embodiments, the tube may comprise one or more cuts configured to affect the flexibility and/or stiffness of the tube. In some embodiments, the one or more cuts extend through the thickness of the tube to form one or more cuts that provide openings, apertures, or voids. In some embodiments, the one or more cuts comprise cuts that do not extend through the thickness of the tube. For example, the cuts may etch the tube material, remove a layer of the tube material, or alter the surface of the tube. In some embodiments, the tube comprises a portion comprising one or more cuts that extend through the thickness of the tube and a portion of the tube comprising one or more cuts that do not extend through the thickness of the tube. The portion comprising the cuts extending through the tube may be more flexible than the portion comprising the cuts that do not extend through the tube.

In some embodiments, a tube comprises a plurality of cuts. In some embodiments, a cut of the plurality of cuts may be characterized at least in part by a kerf. In some embodiments, the kerf refers to the width of the cut. In some embodiments, the kerf is affected by the width of the cutting element that performed the cut. In a non-limiting example, the kerf of the cut may correspond to the width of the cutting element (e.g., blade width, laser beam width) used to cut through the material. In some embodiments, a catheter comprises one or more layers comprising a plurality of cuts. In some embodiments, at least one of the one or more layers is a tube.

In some embodiments, the kerf is defined by a distance between the edges of the cut. In some embodiments, the kerf of a cut may be affected by the amount of stretch applied to the section of the catheter body where the cut is located. In some embodiments, the kerf provides the distance in a radial direction between the edges of the cut. In some embodiments, the kerf provides the distance in an axial direction between the edges of the cut. In some embodiments, the kerf is measured along the longitudinal direction of the tube, extending from one edge of the cut to the other edge. In some embodiments, a tube comprises a plurality of cuts. In some embodiments, the plurality of cuts are not parallel to each other. In some embodiments, the plurality of cuts are parallel to each other. In some embodiments, the edges of a cut are parallel to each other. In some embodiments, the edges of a cut are not parallel to each other. In some embodiments, the edges of a cut are not parallel to each other when the section of the catheter body where the cut is located is stretched out.

In some embodiments, the cuts within the catheter body alter its mechanical properties, such as flexibility, stiffness, and/or compressibility. In some embodiments, the kerf length and width of each kerf is configured to give a catheter or a portion of a catheter a desired stiffness and/or flexibility. In some embodiments, forming the cuts in the cut tube comprises using an automated cutting mechanism. Wherein the automated cutting mechanism may be programmed to create kerfs of a specific width. In some embodiments, the automated cutting mechanism is configured to accommodate tolerance considerations where necessary.

Provided herein is a catheter assembly wherein an interior catheter, an exterior catheter, or both comprise a catheter body comprising a plurality of first cuts in a first section. In some embodiments, the catheter body comprises a plurality of second cuts in a second section. In some embodiments, the first cuts are different from the second cuts. In some embodiments, the first cuts are wider than the second cuts. In some embodiments, the first cuts are longer than the second cuts. In some embodiments, the first cuts are spaced apart further than the second cuts. In some embodiments, the first cuts are spaced the same as the second cuts. In some embodiments, the catheter body comprises a helical cut pattern. In some embodiments, a catheter body comprises a plurality of cuts, forming a cut catheter body. In some embodiments the plurality of cuts form an interrupted spiral pattern along the length of the catheter body. In some embodiments, the cut catheter body comprises a plurality body segments formed by the plurality of cuts. In some embodiments, the plurality of body segments are connected by bridges formed between interrupted cuts. In some embodiments, the plurality of cuts are not parallel to each other. In some embodiments, one or more of body segments consist of a portion of the tube forming a ring around the circumference of the catheter body. In some embodiments, one or more of the body segments consist of a portion of the catheter body forming a single helical turn. In some embodiments, each of body segments consist of a portion of the tube forming a ring around the circumference of the catheter body. In some embodiments, each of body segments consists of a portion of the catheter body forming a single helix turn. In some embodiments, a body segment may comprise a height, wherein the height of the body segments body segments body segments is equal to the pitch (P) of a helical cut pattern. In some embodiments, the body segments may comprise one or more helical turns. In some embodiments, the cuts may extend completely through the wall of the catheter body. The plurality of cuts may comprise interrupted cuts such that a portion of the catheter body material is between subsequent cuts. In some embodiments, the interrupted spiral pattern may comprise a plurality of bridge elements between adjacent body segments. The bridges may connect a body segment to a proximally adjacent body segment and to a distally adjacent body segment. In some embodiments, the stiffness and/or flexibility of a cut catheter body may be varied along a length of the cut catheter body depending on the cut pattern. For example, increasing the pitch (P) between adjacent body segments can increase the stiffness of the catheter body. This may create a stiffer catheter body which may be used to create a stiffer catheter. Alternatively or in combination, reducing the pitch (P) between adjacent body segments can decrease the stiffness of the catheter body. This may make a more flexible catheter body which may be used to create a more flexible catheter. In some embodiments, the width (W) of the bridges may be increased to increase the stiffness of the catheter body. This may create a stiffer catheter body which may be used to create a stiffer catheter. In some embodiments, the width (W) of the bridges may be reduced to decrease the stiffness of the catheter body. This may make a more flexible catheter body which may be used to create a more flexible catheter. In some embodiments, the kerf (K) width of the cuts may be increased to increase the flexibility of the catheter body. This may create a more flexible catheter body which may be used to create a more flexible catheter. In some embodiments, the catheter body comprises a cut tube.

In some embodiments, the cut tube comprises a proximal and a distal uncut section. In some embodiments, the proximal and the distal uncut section are less than about 0.0020 in. In some embodiments, the proximal and the distal uncut section are more than about 0.0020 in. In some embodiments, the proximal and the distal uncut section are less than about 0.10 in. In some embodiments, the proximal and the distal uncut section are more than about 0.10 in. In some embodiments, the proximal and the distal uncut section are about 0.0001 in, about 0.0002 in, about 0.0003 in, about 0.0004 in, about 0.0005 in, about 0.0010 in, about 0.0015 in, about 0.0020 in, about 0.0030 in, about 0.0040 in, about 0.0050 in, about 0.0060 in, about 0.0070 in, about 0.0080 in, about 0.0090 in, about 0.10 in, about 0.20 in, about 0.30 in, about 0.40 in, or about 0.50 in.

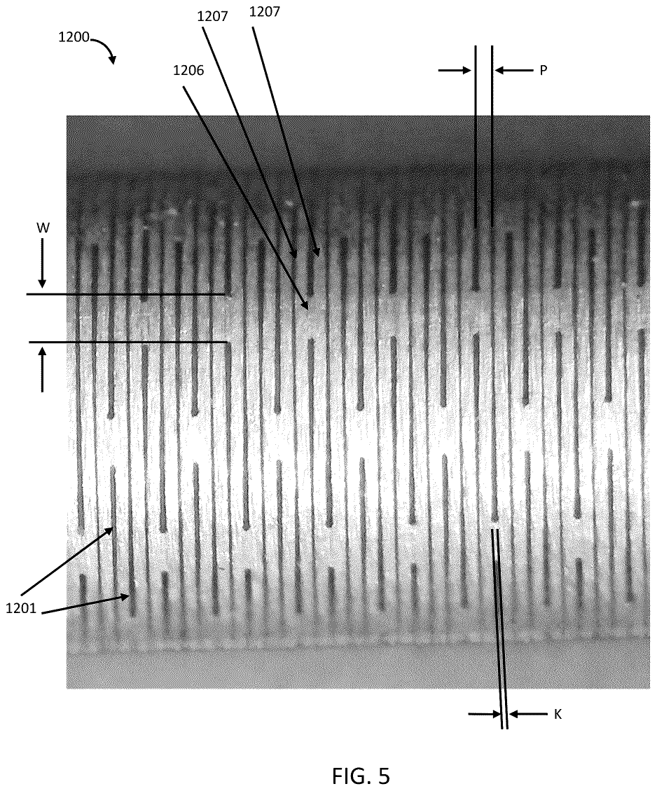

FIG. 5 illustrates an example of a cut tube 1200 comprising a plurality of cuts 1201 forming an interrupted spiral pattern along the length of the cut tube 1200. The interrupted spiral pattern comprises a plurality of helical elements 207 formed by the cuts 1201. In some embodiments, the plurality of cuts 1201 are not parallel to each other. In some embodiments, one or more of helical element 1207 consists of a portion of the tube forming a single helix turn. In some embodiments, each helical element 1207 consists of a portion of the tube forming a single helix turn. In some embodiments, a helical element 1207 may comprise a height, wherein the height of the helical element 1207 is equal to the pitch (P) of said helical element 1207. In some embodiments, a helical element 1207 may comprise one or more helical turns. In some embodiments, the cuts 1201 may extend completely through the wall of the tube 200. The plurality of cuts 1201 may comprise interrupted cuts such that a portion of the tube material is between subsequent cuts. In some embodiments, the interrupted spiral pattern may comprise a plurality of bridge elements 106 between adjacent helical elements 1207. The bridges 106 may connect a helical element 1207 to a proximally adjacent helical element 1207 and to a distally adjacent helical element 1207. In some embodiments, the stiffness and/or flexibility of a cut tube may be varied along a length of the cut tube depending on the cut pattern. For example, increasing the pitch (P) between adjacent helical elements 1207 can increase the stiffness of the cut tube 200. This may create a stiffer tube which may be used to create a stiffer catheter. Alternatively or in combination, reducing the pitch (P) between adjacent helical elements 1207 can decrease the stiffness of the tube. This may make a more flexible tube which may be used to create a more flexible catheter. In some embodiments, the width (W) of the bridges 106 may be increased to increase the stiffness of the tube. This may create a stiffer tube which may be used to create a stiffer catheter. In some embodiments, the width (W) of the bridges 106 may be reduced to decrease the stiffness of the tube. This may make a more flexible tube which may be used to create a more flexible catheter. In some embodiments, the kerf (K) width of the cuts 1201 may be increased to increase the flexibility of the tube. This may create a more flexible tube which may be used to create a more flexible catheter.

In some embodiments, the catheter body comprises a tube. In some embodiments, the catheter body comprises a cut tube. In some embodiments, the catheter body comprises a laser cut tube (LCT). In some embodiments, forming the laser cut tube comprises cutting a tube with a laser. In some embodiments, the tube is a metal hypotube. In some embodiments, the laser removes material with a characteristic width, or kerf (K). The kerf may be dependent on the laser system used, optics in the cutting beam forming, part setup when cutting or a combination thereof. In some embodiments, the cuts are formed by moving the laser along a line. This can allow for a faster cutting process wherein the laser does not need to back track or change speed during cutting. The laser may be interrupted or pulsed to form interrupted cuts. In some embodiments, a cut is formed by a single pass of the laser. In some embodiments, a kerf of a cut is substantially equal to the width of the laser. In some embodiments, a kerf that is substantially equal to the width of the laser is referred to as a laser kerf or a laser cut kerf. In some embodiments, the laser kerf is greater than 0.0015″. In some embodiments, the laser kerf is less than 0.0005″. In some embodiments, a laser kerf is about 0.0005″ to about 0.0015″.

The alterations of flexibility and/or stiffness across the length of the tube may be achieved in numerous ways. In some embodiments, the cut pattern variables (pitch, kerf, cut length, pitch angle) may be varied to affect the flexibility and/or stiffness. In some embodiments, the configurations of one or more cut patterns may be varied to affect the flexibility and/or stiffness. The flexibility and/or stiffness of a tube may be configured to control the flexibility and/or stiffness of a catheter. In some embodiments, a cut tube may comprise one or more sections having different cut patterns along the length of the cut tube. The cut tube may be continuous or discontinuous along the length of the tube. In some embodiments, the cut tube may comprise 1, 2, 3, 4, 5, 6, 7, . . . , or n cut sections along the length of the tube. The cut sections may be continuous or interrupted. In some embodiments, each section of the cut tube may comprise a constant cut pattern. In some embodiments, one or more sections of the cut tube may comprise different cut patterns. In some embodiments, one or more aspects of the cut patterns may be varied e.g., the kerf or pitch. For example, each section of the cut tube may comprise a variable pitch pattern within a section. In some embodiments, a spiral-cut section may have a constant pitch. In some embodiments, the pitch may range from about 0.05 mm to about 10 mm, e.g., about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, etc. In some embodiments, the pitch may be varied within a section. In some embodiments, one or more sections comprise the same or different pitches. In some embodiments, the width or kerf of the cuts of the cut pattern may be varied along the length of the cut tube. In some embodiments, the kerf width ranges from about 0.5 microns to about 100 microns. In some embodiments, the kerf width is less than 0.5 micron. In some embodiments, the kerf width is greater than 100 microns. In some embodiments, the cut may be in a spiral pattern.

In some embodiments, each cut has a height-to-width aspect ratio. In some embodiments, the height-to-width aspect ratio is between about 1:1 to about 50:1. In some embodiments, the height-to-width aspect ratio is 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, or 10:1. In some embodiments, the height-to-width aspect ratio is 10:1, 11:1, 12:1, 13:1, 14:1, 15:1, 16:1, 17:1, 18:1, 19:1, 20:1. In some embodiments, the height-to-width aspect ratio is 20:1, 25:1, 30:1, 35:1, 40:1, 45:1, and 50:1.

In some embodiments, the cut pattern has a cut-to-bridge ratio. In some embodiments, the cut-to-bridge ratio is between about 1:1 to about 50:1. In some embodiments, the cut-to-bridge ratio is 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, or 10:1. In some embodiments, the cut-to-bridge ratio is 10:1, 11:1, 12:1, 13:1, 14:1, 15:1, 16:1, 17:1, 18:1, 19:1, 20:1. In some embodiments, the cut-to-bridge ratio is 20:1, 25:1, 30:1, 35:1, 40:1, 45:1, and 50:1.

In some embodiments, an interrupted cut pattern comprises a pitch. The pitch may be measured by the edge-to-edge distance between adjacent cuts. The pitch may be adjusted to provide a desired flexibility for the catheter. In some embodiments, the pitch may range from about 0.05 mm to about 10 mm, e.g., about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, etc. In some embodiments, the pitch is less than about 0.0010 inches (in). In some embodiments, the pitch is greater than about 0.0010 in. In some embodiments, the pitch is between about 0.0010 in to about 0.050 in. In some embodiments, the pitch is between about 0.0010 in to about 0.010 in. In some embodiments, the pitch is smaller than 0.10 in. In some embodiments, the pitch is larger than 0.010 in. In some embodiments, the pitch is at least about 0.001 in, about 0.002 in, about 0.003 in, about 0.004 in, about 0.005 in, about 0.006 in, about 0.007 in, about 0.008 in, about 0.009 in, about 0.010 in, about 0.011 in, about 0.012 in, about 0.013 in, about 0.014 in, about 0.015 in, about 0.016 in, about 0.017 in, about 0.018 in, about 0.019 in, about 0.020 in, about 0.021 in, about 0.022 in, about 0.023 in, about 0.024 in, about 0.025 in, about 0.026 in, about 0.027 in, about 0.028 in, about 0.029 in, about 0.030 in about 0.031 in, about 0.032 in, about 0.033 in, about 0.034 in, about 0.035 in, about 0.036 in, about 0.037 in, about 0.038 in, about 0.039 in, about 0.040 in, about 0.041 in, about 0.042 in, about 0.043 in, about 0.044 in, about 0.045 in, about 0.046 in, about 0.047 in, about 0.048 in, about 0.049 in, or about 0.050 in. In some embodiments, the pitch is at most about 0.001 in, about 0.002 in, about 0.003 in, about 0.004 in, about 0.005 in, about 0.006 in, about 0.007 in, about 0.008 in, about 0.009 in, about 0.010 in, about 0.011 in, about 0.012 in, about 0.013 in, about 0.014 in, about 0.015 in, about 0.016 in, about 0.017 in, about 0.018 in, about 0.019 in, about 0.020 in, about 0.021 in, about 0.022 in, about 0.023 in, about 0.024 in, about 0.025 in, about 0.026 in, about 0.027 in, about 0.028 in, about 0.029 in, about 0.030 in about 0.031 in, about 0.032 in, about 0.033 in, about 0.034 in, about 0.035 in, about 0.036 in, about 0.037 in, about 0.038 in, about 0.039 in, about 0.040 in, about 0.041 in, about 0.042 in, about 0.043 in, about 0.044 in, about 0.045 in, about 0.046 in, about 0.047 in, about 0.048 in, about 0.049 in, or about 0.050 in. In some embodiments, one or more different sections of the catheter can have different pitches to vary the flexibility along the catheter length.

In some embodiments, an interrupted cut pattern may comprise a certain number of cuts for each turn or rotation. In some embodiments, there may be at least 2, 2.5. 3.5, 4, 4.5, 5, 5.5, 6, 6.5, 7, 7.5, 8, 9, or 10 cuts per turn. In some embodiments, there may be at most 2, 2.5. 3.5, 4, 4.5, 5, 5.5, 6, 6.5, 7, 7.5, 8, 9, or 10 cuts per turn. In some embodiments, there are 2, 2.5. 3.5, 4, 4.5, 5, 5.5, 6, 6.5, 7, 7.5, 8, 9, or 10 cuts per turn.

In some embodiments, the flexibility of the catheter may be altered by changing the size of the kerf. In some embodiments, the flexibility of the cut tube may be altered by changing the size of the kerf. In some embodiments, a cut tube having wider kerfs is more flexible than a cut tube having thinner kerfs.

In some embodiments, kerfs that are wider than the width of the cutting element may be desired. In some embodiments, it may be challenging to make kerfs (K) larger than the width of cutting element (e.g., laser beam) being used. While it may be possible to use laser cutting for making cuts with a kerf wider than the laser width, this may require interpolating a closed loop path for each cut. This may increase manufacturing time (and resulting part cost) as the motion control of the laser system may accelerate and decelerate for each path interpolation. In some cases, laser kerfs are too thin to provide the desired flexibility to a cut tube. In some cases, a cut tube comprising laser cut kerfs may have a limited ductility and/or flexibility. In some cases, the laser kerf of 0.0005″-0.0015″ is too thin to provide the required flexibility required for traversing tortuous anatomy. In some embodiments, the cut filler at least partially fills the cut. In some cases, laser cut kerfs are too thin to allow for the cut filler to fill in the cut. In some cases, the inadequate filling of the cut by the cut filler may result in delamination of an outer jacket and an inner liner from the cut tube, because the cut filler is unable to pass through the kerfs and bond to the outer jacket and the inner liner. In some cases, the catheter having such thin cuts may not be sufficiently flexible to bend as the catheter is advanced through a tortuous anatomy.

In some cases, in order to make a cut tube more flexible, the pitch (P) of the cut pattern may be decreased and/or the widths (W) of the bridges may be decreased. In some embodiments, the pitch (P) of the cut pattern is reduced to improve flexibility of a catheter body to allow for a catheter with sufficient flexibility and/or softness to traverse through torturous anatomy. In some cases, the width (W) of the bridge may be reduced to improve flexibility of a catheter body to allow for a catheter with enough flexibility to traverse through torturous anatomy. This may allow for the highest degree of flexibility and softness allowing the catheter to be advanced through more tortuous anatomy.

In some cases, in order to make a cut tube more flexible, a portion of the cut tube may be stretched. In some cases, stretching a portion of the cut tube may provide a cost-effective method of increasing the kerf width as compared to changing the cutting element or changing the cutting method. In some embodiments, stretching a cut tube will increase the width of the cut kerfs. This may increase the flexibility and/or decrease the stiffness of the cut tube. In some embodiments, stretching a cut tube to increase the width of the cuts may provide a more time efficient and cost-effective method for increasing kerf width and producing a more flexible catheter. In some embodiments, a stretched cut tube may comprise varying degrees of stretch along the length of the cut tube. In some embodiments, the degree of stretch of the stretched cut tube may increase along the length of the cut tube. In some embodiments, the distal end of the cut tube has a higher degree of stretch than the proximal end of the cut tube. In some embodiments the proximal end of a stretched cut tube has no stretch. In some embodiments the proximal end of a stretched cut tube has a minimal degree of stretch such that the size of the kerfs at the proximal end and/or the flexibility of the proximal does not change.

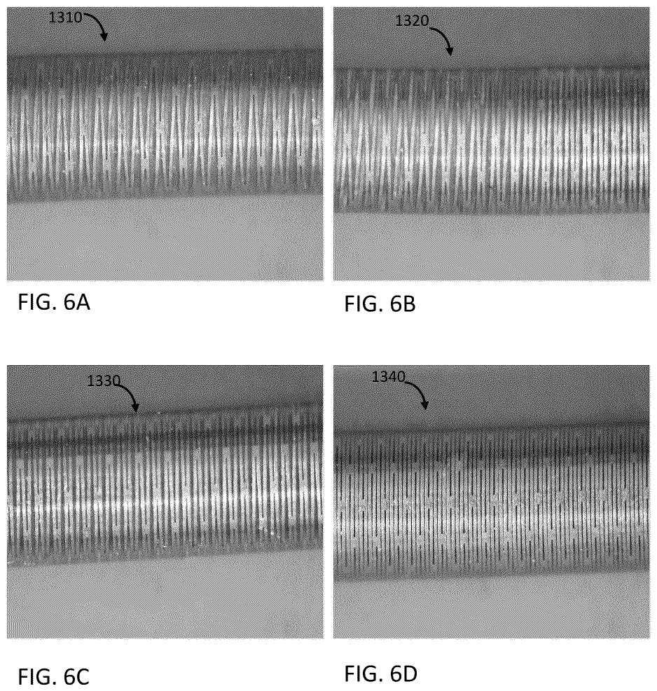

FIGS. 6A-6D illustrate an example of a stretched cut tube having varying degrees of stretch. In some embodiments, the degree of stretch increases along the length of the cut tube. FIG. 6A illustrates a distal portion 1300 of a cut tube. The distal portion 1300 may have the greatest degree of stretch along the length of the cut tube. FIGS. 6B-6C illustrate intermediate sections of the cut tube with decreasing degrees of stretch, wherein cut tube section 1310 may be distal to cut tube section 1320. FIG. 6D illustrates a most proximal section 1330 of the cut tube. In some embodiments, the most proximal section of a cut tube may have the least degree of stretch. In some embodiments, the proximal section of the cut tube 1330 may not be stretched. It can be appreciated in FIG. 6A-6D that varying degrees of stretch may be applied to modify both the bonding between the outer jacket and the liner as well as the catheter stiffness.

In some embodiments, a cut tube comprising a plurality of cuts is configured to be stretched, wherein one or more of the plurality of cuts changes from a first kerf width to a second kerf width. Stretching the cut tube may cause a center width of the cuts (and kerfs) of the cut tube to increase. For example, stretching the cut tube causes a distance of the center of a cut to be wider than the width of the first kerf width formed by the cut. In some embodiments, the stretched cuts may be widest at a center of the cut and narrowest at the ends of the cut. In some embodiments, the cuts narrow from a widest point at the center of the cut to a narrowest point at each end of the cut. In some embodiments, the width at the narrowest point of the cut is substantially equal to the initial kerf width. In some embodiments, one or more stretched cuts may have a widest point at the end of the cut and a narrowest point at the middle of the cut. In some embodiments, one or more stretched cuts may form an hourglass shape. In some embodiments, one or more stretched cuts may have a consistent width along the length of the cut.

In some embodiments, a first kerf width is stretched longitudinally to a second kerf width. In some embodiments, the second kerf width is stretched to at least about 1.01×, 1.05×, 1.1×, 1.2×, 1.3×, 1.4×, 1.5×, 1.6×, 1.7×, 1.8×, 1.9×, 2×, 3×, 4×, 5×, 6×, 7×, 8×, 9×, or 10×. In some embodiments, the second kerf width is stretched to at most about 1.01×, 1.05×, 1.1×, 1.2×, 1.3×, 1.4×, 1.5×, 1.6×, 1.7×, 1.8×, 1.9×, 2×, 3×, 4×, 5×, 6×, 7×, 8×, 9×, or 10×. In some embodiments, the second kerf width is between about 1.01× to about 10× the first cut width. In some embodiments, the second kerf width is between about 1.1× to about 5× the first kerf width. In some embodiments, the second kerf width is between about 1.1× to about 2× the first kerf width.

In some embodiments, after a cut tube has been stretched to open one or more kerfs, an outer layer may be laminated to an inner layer of the catheter. This may help preserve the stretched configuration of the cut tube. In some embodiments, the outer layer comprises an outer jacket of the catheter. In some embodiments, the inner layer comprises a catheter liner. In some embodiments the inner layer comprises a strike layer. In some embodiments, laminating the outer layer of the catheter to the inner layer during assembly holds the structure and configuration of a stretched cut tube. This may allow the stretched cut tube to maintain the wider (stretched) kerf widths after assembly. In some embodiments, stretching the cut tube to open the cuts may improve lamination between an outer layer and inner layer of the catheter. For example, a distal section of the cut tube having the greatest degree of stretch exhibits the best lamination between a liner and outer jacket of the catheter because the wider kerfs provide more contact between the outer and inner layers sandwiching the cut tube. In some embodiments, the portion of the catheter comprising the most stretched portion of the cut tube exhibits excellent lamination between outer jacket and the liner and is also more flexible than a catheter built from the same materials but not stretched. In some embodiments, varying degrees of stretch may be applied to modify both the bonding between the outer jacket and the liner as well as the catheter stiffness. In some embodiments, the degree of stretch of the cut tube gets higher toward the distal end of the catheter. In some embodiments, the outer layer may comprise a cut filler material. In some embodiments, the cut filler material is on an inner facing surface of the outer jacket. In some embodiments, the cut filler material is configured to fill in the kerfs of the cut tube. In some embodiments, the cut filler at least partially fills the cut.

Described herein are methods, devices, and systems configured for variable flexibility and/or stiffness of catheters. In some embodiments, a catheter is configured to have variable stiffness and/or flexibility along the length of the catheter. In some embodiments, the variable stiffness catheter comprises a tube extending at least part of the length of the catheter. In some embodiments, the variable stiffness catheter comprises a tube extending the full length of the catheter or substantially the full length of the catheter. In some embodiments, the tube is configured to affect the flexibility and/or stiffness of the variable stiffness catheter. In some embodiments, the tube is a cut tube. In some embodiments, the variable stiffness catheter comprises a cut tube configured to have a variable stiffness along the length of the cut tube. In some embodiments, the stiffness and/or flexibility of the cut tube is varied by varying the kerf width of the cuts along the length of the cut tube. In some embodiments, at least a portion of the cut tube is stretched, wherein stretching the cut tube alters the stiffness and/or flexibility of the cut tube, wherein stretching the cut tube increases the flexibility of the cut tube. In some embodiments, the entire length of the cut tube is stretched. In some embodiments, a cut tube comprises a first stretched portion configured to have a first stiffness and flexibility. In some additional embodiments, the cut tube may comprise one or more additional stretched portions. The one or more additional stretched portion may be configured to have flexibility and/or stiffness that is different than the first portion. In some embodiments, the one or more additional stretched portions may comprise one or more stretched portions having a stiffness and/or flexibility that is the same as the first stretched portion. In some embodiments, a cut tube comprises two or more stretched portions where in each portion of the cut tube is configured to have a different stiffness and flexibility. In some embodiments, a cut tube comprises a plurality of stretched portions wherein the stiffness and flexibility alternate between adjacent portions. In some embodiments, a cut tube may comprise a distal stretched portion. The distal stretched portion may be more flexible than a proximal portion of the cut tube. In some embodiments, the cut tube may be more flexible at a distal end of the cut tube than a proximal end of the cut tube. In some embodiments, the flexibility of the cut tube may increase at a consistent rate from the proximal end of the cut tube to a distal end of the cut tube. In some embodiments, the flexibility of the cut tube may increase at a variable rate from approximately the cut tube to a distal end of the cut tube. In some embodiments, the rate of change in the flexibility increases as you move distally along the length of the cut tube. In some embodiments, the rate of change in the flexibility decreases as you move distally along the length of the cut tube. In some embodiments, by stretching the cut tube, the size of the kerf is increased (or opened) such that the kerf width is greater than the original kerf width. In some embodiments, the flexibility and/or stiffness of the cut tube is based at least in part on how far the cut tube is stretched.

In some embodiments, heat is applied to the cut tube. In some embodiments, heat is applied to the cut and stretched tube. In some embodiments, a cut tube is stretched and then heated in the stretched configuration. In some embodiments, the application of heat aids in heat setting the cut tube. In some embodiments, a stretched cut tube may be set in place in the stretched configuration by the application of heat without applying a stretch. In some embodiments, a stretched cut tube may keep its stretched configuration after the application of heat without applying a stretch. In some embodiments, heat setting of a cut tube in the stretched configuration may make the assembly of the catheter easier. In some embodiments, the heat setting may make application of the kerf filler, outer jacket, and/or inner liner easier than without the heat setting by keeping the cut tube in the stretched configuration without any stretching applied. In some embodiments, heat setting of a cut tube in the stretched configuration may reduce or eliminate the elastic energy stored in the stretched cut tube. In some embodiments, reducing or eliminating elastic energy may improve mechanical stability of the catheter. In some embodiments, reducing or eliminating elastic energy may facilitate preparation of the catheter. In some embodiments, reducing or eliminating elastic energy may make it easier to handle the cut tube. In some embodiments, heat setting reduces work hardening caused by plastic deformation. In some embodiments, heat setting improves mechanical stability of the catheter.

In some embodiments, a cut tube is heated and then cooled. In some embodiments, a cut tube is heated and then cooled rapidly. In some embodiments, cooling is by an air cool or water quench or a combination thereof.

In some embodiments, heat is applied to the cut tube for at least about 0.1, 0.5, 1, 5, 10, 15, 20, 25, 30, 35, 40, 45, 50, 55, or 60 minutes. In some embodiments, heat is applied to the cut tube for at most about 1, 5, 10, 15, 20, 25, 30, 35, 40, 45, 50, 55, 60, 70, 80, 90, 100, 110, or 120 minutes. In some embodiments, heat is applied to the cut tube for ranges from about 0.5 to about 120 minutes, about 1 to about 60 minutes, about 1 to about 45 minutes, about 1 to about 30 minutes, about 1 to about 20 minutes, or about 1 to about 10 minutes.

In some embodiments, heat applied to the cut tube is at least about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, heat applied to the cut tube is at most about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, heat applied to the cut tube ranges from about 100° C. to about 1500° C., about 100° C. to about 1000° C., about 200° C. to about 800° C., about 300° C. to about 700° C., about 500° C. to about 1500° C., about 700° C. to about 1500° C., or about 800° C. to about 1300° C.

In some embodiments, the cut tube is heat to at least about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, the cut tube is heat to at most about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, the cut tube is heat to a range of about 100° C. to about 1500° C., about 100° C. to about 1000° C., about 200° C. to about 800° C., about 300° C. to about 700° C., about 500° C. to about 1500° C., about 700° C. to about 1500° C., or about 800° C. to about 1300° C.

In some embodiments, a cut tube comprising NiTi can be heat set. In some embodiments, a cut tube comprising NiTi maintains its superelastic property after heat setting. In some embodiments, a cut tube comprising NiTi that is heat set can bend when a load is applied. In some embodiments, a cut tube comprising NiTi may be stretched to deformations within its superelastic plateau. In some embodiments, a cut tube comprising NiTi is heat set after being stretched to deformations within its superelastic plateau. In some embodiments, a cut tube comprising NiTi may be heat set at a temperature range of about 400° C. to about 600° C. for 1 to 30 minutes followed by a rapid temperature drop (e.g., a quench). In some embodiments, a cut tube comprising NiTi may be heat set at a temperature of about 500° C. for 1 to 30 minutes followed by a rapid temperature drop (e.g., a quench). In some embodiments, a cut tube comprising NiTi may be heat set at a temperature range of about 400° C. to about 600° C. In some embodiments, a cut tube comprising NiTi may be heat set for 1 to 30 minutes.

In some embodiments, a cut tube comprising stainless steel may be heat set at a temperature of about 900° C. to about 1300° C. for 1 to 60 minutes and then cooled. In some embodiments, a cut tube comprising stainless steel may be heat set at a temperature of about 1040° C. to about 1175° C. for 1 to 30 minutes and then cooled.

In some embodiments, the catheter comprises a tip at a distal end of the catheter body. In some embodiments, the tip is a flat tip. In some embodiments, the flat tip comprises a flat distal end. In some embodiments, the tip comprises one or more distal extensions. In some embodiments, the distal extension is flexible. In some embodiments, the tip does not have any distal extensions. In some embodiments, the tip comprises one or more holes. In some embodiments, the tip is flexible. In some embodiments, the tip is made of a flexible material. In some embodiments, the holes aids in providing flexibility to the tip.

### Cutting Methods

Methods for forming a cut tube as described herein may comprise one or more methods for mechanically cutting a pattern into a tube. In some embodiments, cutting the pattern into the tube comprises one or more non-mechanical cutting methods. In some embodiments, the notches, holes or cuts may be formed in the tube using a laser (solid-state, femtosecond laser, or YAG laser, for example), electrical discharge (electrical discharge machining (EDM)), plasma blade, pressurized fluid jet (waterjet), a lathe, a saw, a blade, chemical etching, photo-etching, or a combination thereof. In some embodiments, methods of cutting a pattern into a tube may comprise laser cutting, plasma cutting, hydrocutting, thermal cutting, mechanical cutting, or a combination thereof.

In some embodiments, a tube comprises one material. In some embodiments, the tube comprises two or more materials. In some embodiments, the tube comprises a metal, metal alloy, polymer, or combination thereof. The tube may comprise nitinol, stainless steel, or a combination thereof. In some embodiments, the tube comprises stainless steel, nickel titanium (nitinol), or polymers. In some embodiments, the tube comprises, super elastic NiTi alloy super elastic nickel titanium, shape memory nickel titanium, TiNi, nickel titanium, NiTiHf, NiTiPd, NiMnGa, stainless steel. In some embodiments, the stainless steel may comprise Stainless having an SAE grade in the 200, 300, or 400 series, precipitation hardened (PH) stainless steel, other spring steel or other high tensile strength material or other biocompatible metal material. In some embodiments, the material of a tube comprises superelastic or shape memory metal (e.g., nickel titanium), Alternatively or in combination, the material of a tube comprises stainless steel.

### Catheter Layers

In some embodiments, the one or more layers of the outer jacket may comprise an inner layer and an outer layer. In some embodiments, the liner comprises one or more layers. In some embodiments, the liner comprises a strike layer. In some embodiments, the multiple layers of the outer jacket may allow the inner layer to be incorporated in between the catheter body (such as LCT) and the outer layer. In some embodiments, the outer layer may be configured to lie on top of the inner layer. Alternatively, or in combination, the outer layer may be configured to lie on top of the catheter body. In some embodiments, the inner layer comprises a first polymer. In some embodiments, the outer material comprises a second polymer. In some embodiments, the second polymer is configured to make the first polymer easier to handle. In some embodiments, the inner material may comprise a soft tacky material. In some embodiments, the inner layer may comprise a cut filler material also referred to as a filler material, configured to fill the voids (e.g., kerfs) of the catheter body. In some embodiments, the filler material is configured to fill the kerfs of a cut catheter body. In some embodiments, the filler material is configured to elongate and compress while the catheter is stretched. In some embodiments, using a softer, more elastic filler material will result in a more flexible catheter. In some embodiments, the outer layer of the outer jacket may be configured to be harder (i.e., more rigid or less flexible) than the filler material. In some embodiments, the material of the outer layer may be configured to improve tensile strength of the catheter, reduce friction properties of the outer layer, and/or improve compatibility with one or more coatings applied to the catheter. In some embodiments, having a multiple layer catheter makes catheter assembly easier, because the more flexible materials of the inner layer may be very tacky and/or difficult to handle. In some embodiments, the filler material may comprise a non-flowing soft material. In some embodiments, the inner layer comprises a gel material or rubber material. In some embodiments, the harder outer layer may be configured to make the inner layer easier to handle and load over the catheter body and liner assembly. In some embodiments, the filler material may contact the outer jacket. In some embodiments, an outer layer is configured to be laminated to the catheter body. In some embodiments, an outer layer is configured to be laminated to the liner. In some embodiments, an outer layer is configured to be laminated in combination with the inner layer.

In some embodiments, the outer layer of the catheter may be formed of a stiffer material than the inner layer. In some embodiments, the outer layer material may be configured to provide improved tensile strength of the catheter assembly, reduce friction properties of the outer layer, improve compatibility with any coatings applied to the catheter, and/or a combination thereof. In some embodiments, an outer layer is configured to be laminated to the catheter assembly in combination with the inner layer. In some embodiments, a stiffer outer layer is configured to make catheter assembly easier, because the very soft materials of the inner layer may be very tacky or otherwise difficult to handle. In some embodiments, the stiffer outer layer may make the tube easier to handle and load over the LCT and liner assembly.

In some embodiments, the filler material is configured to fill in the space of the cuts. In some embodiments, the filler material is a viscous liquid. In some embodiments, the filler material comprises a non-Newtonian fluid. In some embodiments, the filler material is bonded to the outer jacket. In some embodiments, the filler material contacts the liner. In some embodiments, the filler material is bonded to the liner. In some embodiments, the liner comprises a strike layer configured to facilitate bonding of the liner to the filler material. In some embodiments, a surface of the liner is modified to facilitate bonding between the liner and the filler material.

In some embodiments, the inner layer of the outer jacket comprises a material having a durometer of at least 0.1 Shore 00, 0.5 Shore 00, 1.0 Shore 00, 2.0 Shore 00, 3.0 Shore 00, 4.0 Shore 00, 5.0 Shore 00, 6.0 Shore 00, 7.0 Shore 00, 8.0 Shore 00, 9.0 Shore 00, 10 Shore 00, 20 Shore 00, 30 Shore 00, 40 Shore 00, 50 Shore 00, 60 Shore 00, 70 Shore 00, 80 Shore 00, 90 Shore 00, 0.1 Shore A, 0.5 Shore A, 1.0 Shore A, 2.0 Shore A, 3.0 Shore A, 4.0 Shore A, 5.0 Shore A, 6.0 Shore A, 7.0 Shore A, 8.0 Shore A, 9.0 Shore A, 10 Shore A, 11 Shore A, 12 Shore A, 13 Shore A, 14 Shore A, 15 Shore A, 16 Shore A, 17 Shore A, 18 Shore A, 19 Shore A, 20 Shore A, 21 Shore A, 22 Shore A, 23 Shore A, 24 Shore A, 25 Shore A, 26 Shore A, 27 Shore A, 28 Shore A, 29 Shore A, 30 Shore A, 31 Shore A, 32 Shore A, 33 Shore A, 34 Shore A, 35 Shore A, 36 Shore A, 37 Shore A, 38 Shore A, 39 Shore A, 40 Shore A, 41 Shore A, 42 Shore A, 43 Shore A, 44 Shore A, 45 Shore A, 46 Shore A, 47 Shore A, 48 Shore A, 49 Shore A, 50 Shore A, 51 Shore A, 52 Shore A, 53 Shore A, 54 Shore A, 55 Shore A, 56 Shore A, 57 Shore A, 58 Shore A, 59 Shore A, 60 Shore A, 61 Shore A, 62 Shore A, 63 Shore A, 64 Shore A, 65 Shore A, 66 Shore A, 67 Shore A, 68 Shore A, 69 Shore A, 70 Shore A, 71 Shore A, 72 Shore A, 73 Shore A, 74 Shore A, 75 Shore A, 76 Shore A, 77 Shore A, 78 Shore A, 79 Shore A, 80 Shore A, 81 Shore A, 82 Shore A, 83 Shore A, 84 Shore A, 85 Shore A, 86 Shore A, 87 Shore A, 88 Shore A, 89 Shore A, 90 Shore A. In some embodiments, the inner layer of the outer jacket comprises a material having a durometer of at most 0.1 Shore 00, 0.5 Shore 00, 1.0 Shore 00, 2.0 Shore 00,3.0 Shore 00, 4.0 Shore 00, 5.0 Shore 00, 6.0 Shore 00, 7.0 Shore 00, 8.0 Shore 00, 9.0 Shore 00, 10 Shore 00, 20 Shore 00, 30 Shore 00, 40 Shore 00, 50 Shore 00, 60 Shore 00, 70 Shore 00, 80 Shore 00, 90 Shore 00, 0.1 Shore A, 0.5 Shore A, 1.0 Shore A, 2.0 Shore A, 3.0 Shore A, 4.0 Shore A, 5.0 Shore A, 6.0 Shore A, 7.0 Shore A, 8.0 Shore A, 9.0 Shore A, 10 Shore A, 11 Shore A, 12 Shore A, 13 Shore A, 14 Shore A, 15 Shore A, 16 Shore A, 17 Shore A, 18 Shore A, 19 Shore A, 20 Shore A, 21 Shore A, 22 Shore A, 23 Shore A, 24 Shore A, 25 Shore A, 26 Shore A, 27 Shore A, 28 Shore A, 29 Shore A, 30 Shore A, 31 Shore A, 32 Shore A, 33 Shore A, 34 Shore A, 35 Shore A, 36 Shore A, 37 Shore A, 38 Shore A, 39 Shore A, 40 Shore A, 41 Shore A, 42 Shore A, 43 Shore A, 44 Shore A, 45 Shore A, 46 Shore A, 47 Shore A, 48 Shore A, 49 Shore A, 50 Shore A, 51 Shore A, 52 Shore A, 53 Shore A, 54 Shore A, 55 Shore A, 56 Shore A, 57 Shore A, 58 Shore A, 59 Shore A, 60 Shore A, 61 Shore A, 62 Shore A, 63 Shore A, 64 Shore A, 65 Shore A, 66 Shore A, 67 Shore A, 68 Shore A, 69 Shore A, 70 Shore A, 71 Shore A, 72 Shore A, 73 Shore A, 74 Shore A, 75 Shore A, 76 Shore A, 77 Shore A, 78 Shore A, 79 Shore A, 80 Shore A, 81 Shore A, 82 Shore A, 83 Shore A, 84 Shore A, 85 Shore A, 86 Shore A, 87 Shore A, 88 Shore A, 89 Shore A, 90 Shore A.

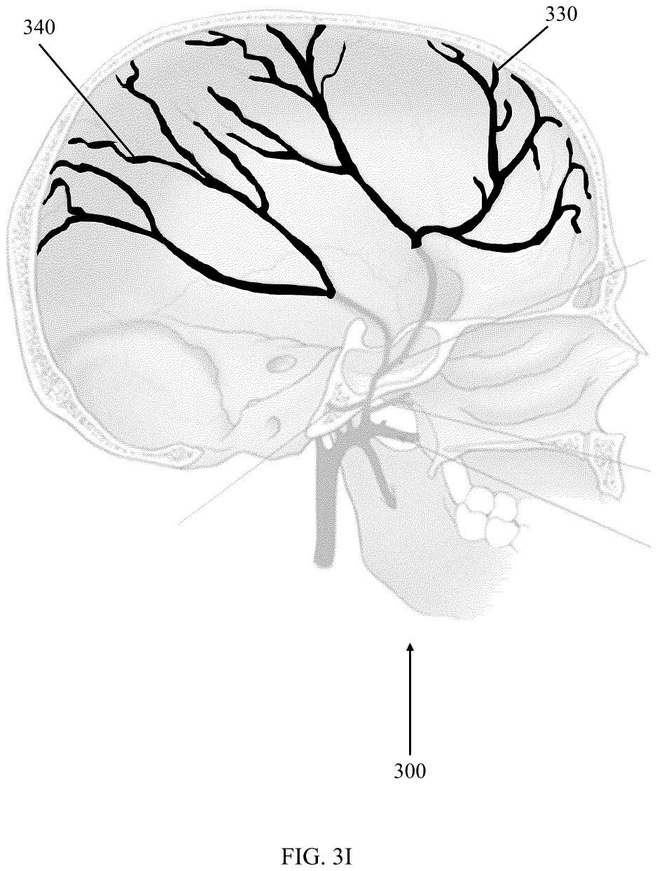

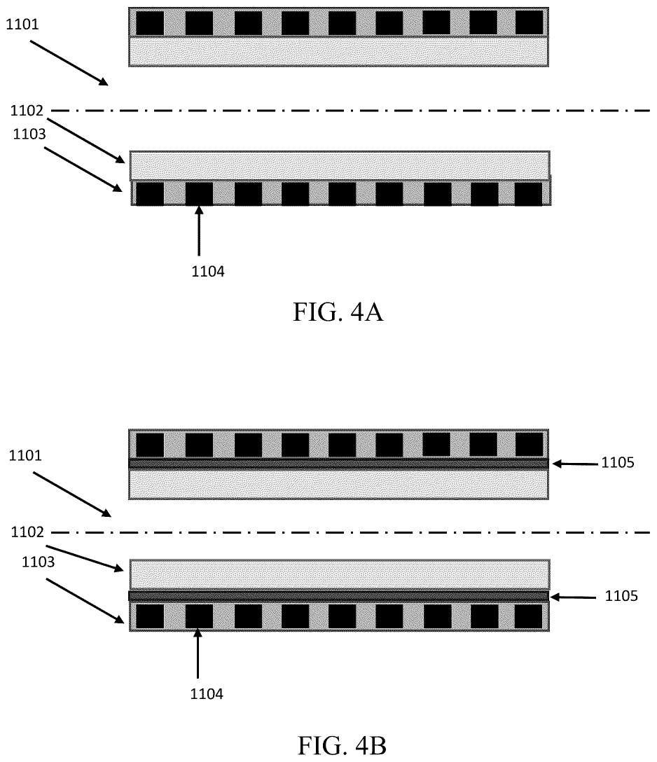

In some embodiments, a catheter as described herein comprises a catheter body, wherein the catheter body is formed of composite structures that have different elements with specific functions to optimize catheter performance. As illustrated in FIG. 4, a catheter may comprise an inner layer 1102, and outer layer 1103 (or outer jacket), and a catheter body 1104. The inner-most layer 1102 of the catheter (meaning, the layer adjacent to the central lumen 1101), may be a liner. In some embodiments, the liner 1102 may comprise a polymer having a low coefficient of friction. The liner material may comprise pebax (friction modified or natural), PTFE, or PE. In some embodiments, a liner material may be selected based on a coefficient of friction with other items that may be tracked through the central lumen of the catheter and/or bondability to an outer jacket 1103. In some embodiments, a liner 1102 may comprise one or more surface modification. In some embodiments, the surface modifications of the liner 1102 may comprise one or more of a chemical modification, textural modification, or plasma modification. In some embodiments, the surface modification may be configured to improve the bondability of the liner 1102 to the jacket material 1103. In some embodiments, a catheter may further comprise strike layer 1105, as illustrated in FIG. 4B. In some embodiments, the strike layer 1105 is configured to improve bondability between the liner 1102 and jacket 1103. In some embodiments, the strike layer 1105, may be a strike layer 1105. In some embodiments, the strike layer is configured to improve bondability between the liner and jacket.

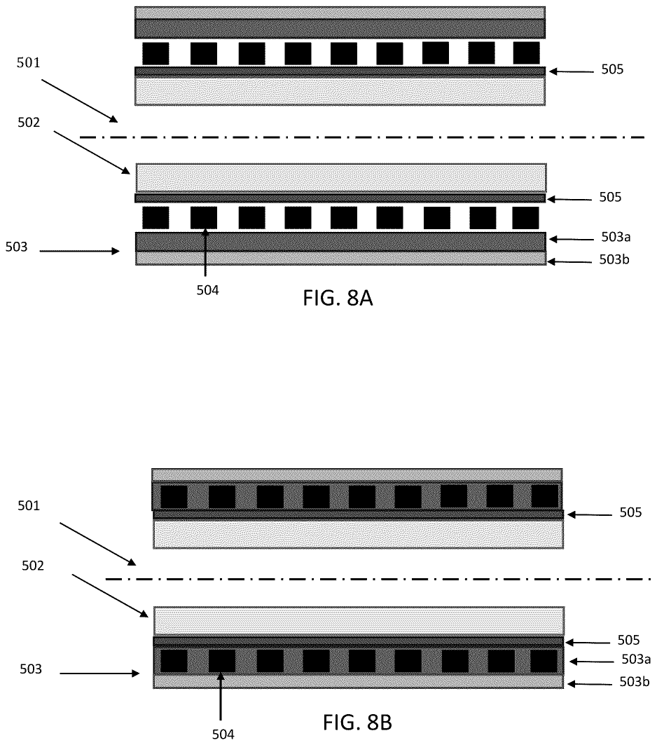

In some embodiments, the catheter comprises one or more layers similar to the catheter shown in FIG. 4. In some embodiments, having a multiple layer catheter as shown in FIGS. 8A and 8B, makes catheter assembly easier, because the more flexible materials of the inner layer may be very tacky and/or difficult to handle. FIGS. 8A and 8B illustrate an embodiment of a catheter comprising an inner liner 502, a strike layer 503, a catheter body 504, and a multi-layer outer jacket 503. In some embodiments, catheter body comprises an outer jacket 503. In some embodiments, the outer jacket 503 comprises an inner layer 503a and an outer layer 503b. FIG. 8A illustrates an example of the multilayer catheter cross-section comprising an inner liner 502, a strike layer 503, a catheter body 504, and a multi-layer outer jacket 503, wherein the layers are assembled, but not yet laminated together. FIG. 8B illustrates the multilayer catheter cross-section comprising the layers of the catheter illustrated in FIG. 8A, after lamination. In some embodiments, the catheter comprises a liner 502. In some embodiments, the liner 502 further comprises a strike layer 505. In some embodiments, the multiple layers of the outer jacket 503 may allow the inner layer 503a to be incorporated in between the catheter body 504 (such as LCT) and the outer layer 503b. In some embodiments, the outer layer 503b may be configured to lie on top of the catheter body 504. In some embodiments, the inner layer 503a may comprise a cut filler material also referred to as a filler material, configured to fill the voids (e.g., kerfs) of the catheter body. In some embodiments, the filler material is configured to fill the kerfs of the catheter body. In some embodiments, the filler material is configured to elongate and compress while the catheter is stretched. In some embodiments, using a softer, more elastic filler material will result in a more flexible catheter. In some embodiments, the outer layer 503b of the outer jacket 503 may be configured to be harder (i.e., more rigid or less flexible) than the filler material. In some embodiments, the material of the outer layer 503b may be configured to improve tensile strength of the catheter, reduce friction properties of the outer layer, and/or improve compatibility with one or more coatings applied to the catheter. In some embodiments, having a multiple layer tube as shown in FIGS. 8A-B may make catheter assembly easier, because the more flexible materials of the inner layer 503a may be very tacky and/or difficult to handle. In some embodiments, the harder outer layer 503b may be configured to make the inner layer 503a easier to handle and load over the catheter body and liner assembly. In some embodiments, the filler material may contact the outer jacket.

In some embodiments, the outer layer 503b of the catheter, may be formed of a stiffer material than the inner layer 503a. In some embodiments, the outer layer 503b material may be configured to provide improved tensile strength of the catheter assembly, reduce friction properties of the outer layer, improve compatibility with any coatings applied to the catheter, and/or a combination thereof. In some embodiments, an outer layer 503b is configured to be laminated to the catheter assembly in combination with the inner layer 503a. In some embodiments, a stiffer outer layer 503b is configured to make catheter assembly easier, because the very soft materials of the inner layer 503a may be very tacky or otherwise difficult to handle. In some embodiments, the stiffer outer layer 503b may make the tube 503 easier to handle and load over the LCT and liner 502 assembly.

In some embodiments, the filler material is configured to fill in the space of the laser cuts. In some embodiments, the filler material is a viscous liquid. In some embodiments, the filler material comprises a non-Newtonian fluid. In some embodiments, the filler material 503a is bonded to the outer jacket. 503 In some embodiments, the filler material 503a contacts the liner 502. In some embodiments, the filler material 503a is bonded to the liner 502. In some embodiments, the liner 502 comprises a strike layer 505 configured to facilitate bonding of the liner 502 to the filler material 503a. In some embodiments, a surface of the liner 502 is modified to facilitate bonding between the liner 502 and the filler material 503a.

In some embodiments, a catheter as described herein comprises a catheter body, wherein the catheter body is formed of composite structures that have different elements with specific functions to optimize catheter performance. In some embodiments, a catheter may comprise an inner layer, an outer layer (or outer jacket), and a catheter body. The inner-most layer of the catheter (meaning, the layer adjacent to the central lumen), may be a liner.

In some embodiments, the liner may comprise a polymer having a low coefficient of friction. The liner material may comprise Pebax (friction modified or natural), PTFE, or PE or a combination thereof. In some embodiments, a liner material may be selected based on a coefficient of friction with other items that may be tracked through the central lumen of the catheter and/or bondability to an outer jacket. In some embodiments, a liner may comprise one or more surface modifications. In some embodiments, the surface modifications of the liner may comprise one or more of a chemical modification, textural modification, or plasma modification. In some embodiments, the surface modification may be configured to improve the bondability of the liner to the jacket material. In some embodiments, a catheter may further comprise strike layer. In some embodiments, the strike layer is configured to improve bondability between the liner and jacket. In some embodiments, the strike layer, may be a strike layer. In some embodiments, the strike layer is configured to improve bondability between the liner and jacket. In some embodiments, the strike layer has a thickness of at least about 0.0001 inches. In some embodiments, the strike layer has a thickness of at most about 0.003 inches. In some embodiments, the strike layer has a thickness in the range of about 0.0001 inches to about 0.003 inches. In some embodiments, the strike layer may comprise a polymer that exhibits good bondability to the jacket material. In some embodiments, the strike layer is provided over the liner prior to the catheter. This may provide more assurance that a good bond is established between the liner and jacket. In some embodiments, a catheter comprises a catheter body. The catheter body may be configured to improve the kink resistance of the catheter wall. In some embodiments, the catheter body may be metallic (e.g., stainless steel, superelastic NiTi, or radiopaque materials such as tungsten or platinum/iridium alloy). In some embodiments, the catheter body may be a polymeric material, including but not limited to LCP (liquid crystal polymer), polyimide, PEEK, or nylon, or a combination thereof. The catheter body may take the form of a tubular braid or a coil.

In some embodiments, the catheter body comprises a plurality of cuts. In some embodiments, the plurality of cuts form a cut pattern along at least a portion of the catheter body. In some embodiment, the cut pattern may be a continuous cut pattern. In some embodiments, the cut pattern may be an interrupted cut pattern. In some embodiments, the cut pattern may comprise one or more different cut patterns along one or more portions of the catheter body. In some embodiments, the cut pattern comprises one or more of longitudinal cuts, axial cuts, diagonal cuts, curved cuts, or any other shaped cut. In some embodiments, the plurality of cuts form an interrupted spiral pattern. In some embodiments, the catheter body may provide an improvement over existing reinforcement elements like a coil or braid. The catheter body may be made from the same materials as the traditional reinforcement element materials. In some embodiments, to create the cut pattern of the catheter body, a cutting element is passed over the tube in a helical path. In some embodiments, the cutting element cuts completely through the wall of the catheter body. In some embodiments the cutting element cuts a helical path, wherein the cut catheter body would resemble a coil. In some embodiments, the cutting element is a laser, a plasma blade, a pressurized fluid jet (waterjet), a lathe, a saw, a blade, or any other suitable cutting element.

In some embodiments, the catheter body comprises a cut tube. In some embodiments, the cut tube as described herein, may provide an improvement over existing reinforcement elements like a coil or braid. The tube may be made from the same materials as the traditional reinforcement element materials. In some embodiments, to create the interrupted spiral pattern of the cut tube, a laser is passed over the tube in a helical path. In some embodiments, the laser cuts completely through the wall of the tube support material and cuts the entire helical path, wherein the cut tube would be a structure resembling a coil. In some embodiments, to create the cut pattern of the cut tube, a cutting element is passed over the tube in a helical path. In some embodiments, the cutting element cuts completely through the wall of the cut tube material. In some embodiments the cutting element cuts a helical path, wherein the cut tube would resemble a coil. In some embodiments, the cutting element is a laser, a plasma blade, a pressurized fluid jet (waterjet), a lathe, a saw, a blade, or any other suitable cutting element.

In some embodiments, a catheter comprises at least, an inner liner layer, a catheter body, and an outer jacket layer. In some embodiments, the catheter body comprises a tube. In some embodiments, the liner is bonded to an inner surface of the catheter body. In some embodiments, the liner is bonded to an inner surface of the tube. In some embodiments, the inner surface may be a flat surface. In some embodiments, the outer jacket layer may comprise a polymeric material, including but not limited to HDPE, PTFE, ETFE, FEP, Pebax, nylon, or other polymer material, or combination thereof. In some embodiments, the outer jacket is fused over the catheter body.

In some embodiments, at least a portion of the lumen of the catheter body comprises a polymer liner bonded to the inner wall of the catheter body. In some embodiments, the polymer liner may form a tube. In some embodiments, the catheter body may be configured co-axially within the lumen of the catheter body. In some embodiments, the liner may include at least two polymer layers. In some embodiments, the liner is bonded to the inner wall of the catheter body along the length of the catheter body. In some embodiments, the liner is bonded continuously to the inner wall of the catheter body along the entire length of the catheter body. In some embodiments, the liner is bonded to two or more sections of the inner wall of the catheter body spaced apart along the longitudinal axis of the catheter body. The polymer liner may be bonded to the inner wall of the catheter body in a continuous helical pattern running along at least a portion of the length of the catheter body. The polymer liner may be bonded to the inner wall of the catheter body by melting the polymer to the catheter body at one or more sections of the catheter body. In some embodiments, the surface of the liner facing the lumen is coated with a lubricous material.

In some embodiments, the catheter body is covered by an outer jacket. In some embodiments, the outer jacket may be coated with a lubricious material.

In some embodiments, a proximal section of the tube has less axial flexibility than a distal section of the catheter frame.

In some embodiments, the liner comprises one or more polymer layers. The one or more polymer layers may form a tubular structure. In some embodiments, the one or more layers of the liner comprises one or more of polytetrafluoroethylene (PTFE), ethylene tetrafluoroethylene (ETFE), fluorinated ethylene propylene (FEP), polyoxymethylene, polyether block ester, polyurethane, polypropylene, polyvinylchloride, polyether-ester, ether or ester based copolymers, phthalate and/or other polyester elastomers, polyamide, elastomeric polyamides, block polyamide/ethers, polyether block amide, ethylene vinyl acetate copolymers (EVA), silicones, polyethylene (PE), Marlex high-density polyethylene, Marlex low-density polyethylene, linear low density polyethylene, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR), polysulfone, nylon, nylon-12, perfluoro(propyl vinyl ether) (PFA), ethylene vinyl alcohol, polyolefin, polystyrene, epoxy, polyvinylidene chloride (PVdC), poly(styrene-b-isobutylene-b-styrene), polycarbonates, ionomers, biocompatible polymers. In some embodiments, the one or more layers of the liner comprises mixtures, combinations, copolymers, polymer/metal composites of any of the aforementioned materials. In some embodiments, the liner comprises a liquid crystal polymer (LCP).

In some embodiments, the liner may be disposed within the lumen of the tube and extend from the proximal end of the catheter body to the distal end of the catheter body. In some embodiments, the length of the liner is longer than the length of the catheter body.

In some embodiments, the liner may be partially and/or intermittently bonded or adhered to the catheter body. To further contribute to the overall flexibility of the catheter. In some embodiments, the liner is bonded to the catheter body by heat fusing/melting, adhesives, or other bonding processes. In some embodiments, a strike layer is used to assist improve bonding/attachment between the liner and the catheter body. For example, in a device utilizing a liner constructed from PTFE, a strike layer may comprise a PEBAX® powder coating between the PTFE liner and the catheter body. In some embodiments, bonding the liner to the catheter body comprises heating the catheter body at a temperature sufficient to melt the strike layer, but at a temperature lower than the temperature required to melt the liner. This may allow the melted strike layer to bond the liner to the catheter body. In some embodiments, the strike layer is a tacky material, and the liner is a more rigid material. In some embodiments, the tacky strike layer is configured to fill in the void space of the cuts for a cut tube. The tacky material may be configured to expand and compress with the cuts of the cut tube while bending.

In some embodiments, an outer jacket may comprise nylon, polyether block amide, PTFE, FEP, PFA, PET, PEEK, or combinations thereof. In some embodiments, the outer jacket has a thickness of at least about 0.0005 inches. In some embodiments, the outer jacket has a thickness of at most about 0.010 inches. In some embodiments, the strike layer has a thickness in the range of about 0.0005 inches to about 0.010 inches. In some embodiments, the outer jacket may be configured to provide an atraumatic, protective covering over the catheter body. In some embodiments, the outer jacket is configured to minimize trauma and/or pinching of tissue while advancing the catheter through torturous tissue.

In some embodiments, an outer jacket has a substantially smooth, outer surface. In some embodiments, the outer jacket may include one or more surface modifications to the outer surface. In some embodiments, the outer layer comprises the tube. The tube may comprise a cut pattern or other geometric features that alter the overall flexibility of the catheter assembly.

The outer jacket may be formed from a polymer, laminated to the liner (or strike layer enclosing the catheter body. In some embodiments, the outer jacket comprises a tacky inner layer and the stiffer outer layer. In some embodiments, the inner layer and outer layer are co-extruded over the catheter body. In some embodiments, the inner layer and outer layer are simultaneously extruded. In some embodiments, the inner layer is co-extruded with the outer layer. In some embodiments, the inner layer is co-extruded with the outer layer wherein the outer layer is extruded directly behind the extrusion of the tacky inner layer. This may help control the lamination of the tacky material wherein the immediately following stiffer outer layer controls and contains the tacky material. In some embodiments, the inner layer and the outer layer are co-extruded from a single extrusion nozzle. In some embodiments, the outer layer is configured to be extruded atop the inner layer. In some embodiments, the inner layer and the outer layer are co-extruded from adjacent extrusion nozzles, wherein the outer layer extrusion nozzle directly follows the inner layer nozzle.

In some embodiments, the outer jacket comprises a polymer material. For example, nylon, polyether block amide, PTFE (polytetrafluoroethylene), FEP (fluorinated ethylene propylene), PFA (perfluoroalkoxy alkane), PET (polyethylene terephthalate) or PEEK (polyether ether ketone). In some embodiments, at least a portion of the outer layer of the outer jacket may be coated with a hydrophilic polymer coating configured to improve lubricity and/or trackability of the catheter. For example, the hydrophilic polymer coatings may comprise, polyelectrolyte and/or a non-ionic hydrophilic polymer, where the polyelectrolyte polymer can include poly(acrylamide-co-acrylic acid) salts, a poly(methacrylamide-co-acrylic acid) salts, a poly(acrylamide-co-methacrylic acid) salts, etc., and the non-ionic hydrophilic polymer may be poly(lactams), for example polyvinylpyrollidone (PVP), polyurethanes, homo- and copolymers of acrylic and methacrylic acid, polyvinyl alcohol, polyvinylethers, snapic anhydride based copolymers, polyesters,

[symbol]

The outer jacket may be formed by enclosing a tube wall with a co-extruded polymer of single or multiple layers. In some embodiments, assembling the catheter may comprise heat shrinking the tube or coating the tube frame via a dip coating process. The polymer jacket material may comprise nylon, polyether block amide, PTFE (polytetrafluoroethylene), FEP (fluorinated ethylene propylene), PFA (perfluoroalkoxy alkane), PET (polyethylene terephthalate) or PEEK (polyether ether ketone). Further, a portion of the catheter (or the entire length of the catheter) may be coated with a hydrophilic polymer coating to enhance lubricity and trackability. Hydrophilic polymer coatings can include, but are not limited to, polyelectrolyte and/or a non-ionic hydrophilic polymer, where the polyelectrolyte polymer can include poly(acrylamide-co-acrylic acid) salts, a poly(methacrylamide-co-acrylic acid) salts, a poly(acrylamide-co-methacrylic acid) salts, etc., and the non-ionic hydrophilic polymer may be poly(lactams), for example polyvinylpyrollidone (PVP), polyurethanes, homo- and copolymers of acrylic and methacrylic acid, polyvinyl alcohol, polyvinylethers, snapic anhydride based copolymers, polyesters, hydroxypropylcellulose, heparin, dextran, polypeptides, etc. In some embodiments, a coating to the outer jacket may be applied by a dip coating process or by spraying the coating onto the tube outer and inner surfaces. In some embodiments, the outer jacket is configured to cover the catheter body. In some embodiments, the outer jacket may be coated with a lubricious material. In some embodiments, a proximal section of the catheter body has stiffer outer jacket than a distal section of the catheter body.

In some embodiments, the outer jacket or liner may comprise a lubricious coating or film configured to improve the catheter's ability to be advanced through torturous tissue. In some embodiments, the lubricious coating may comprise silicone or hydrogel polymers or the like.

In some embodiments, manufacturing a catheter described herein comprises laminating one or more materials to a catheter body. In some embodiments, lamination comprises laminating a first layer, onto a catheter body or mandrel. In some embodiments, laminating the first layer onto the catheter body comprises extruding a first material onto the catheter body. In some embodiments, the first material comprises a soft material such as a thermoplastic elastomer, silicone, polyurethane, or other biocompatible materials.

In some embodiments, lamination comprises laminating a second layer, on top of the first layer. In some embodiments, laminating the second layer on top of the first layer comprises extruding a second material onto the first material. In some embodiments, the second material comprises of a stiffer material than the first material. In some embodiments, the second stiffer material may comprise a biocompatible polymer. Inn some embodiments, the second layer is configured to provide structural integrity and kink resistance to the catheter. This may improve the catheter's ability to navigate the tortuous body lumens without buckling, twisting, or bending.

This two-step extrusion process, involving lamination of two distinct material layers, results in a catheter possessing both softness for patient comfort and stiffness for operational efficiency. Extrusion lamination offers advantages over conventional coating or dipping methods, providing uniform material layers and improved bonding between layers. In some embodiments, the lamination of the catheter body may be tailored to achieve desired layer thicknesses and flexibility/stiffness properties, to the specific needs of various catheter applications.

In some embodiments, laminating the catheter body comprises extruding a flowable material over the catheter body. In some embodiments, the flowable material is only flowable under a particular heat and/or pressure. The flowable material may be flowed over the catheter body while in a particular environment configured to allow the material to flow, wherein the material becomes solid after the catheter body is removed from the environment.

In some embodiments, laminating the catheter body comprises laminating a cut catheter body. In some embodiments, the cut catheter body is stretched prior to lamination of the catheter body. Stretching the catheter body may increase the kerf width of one or more cuts of the cut catheter body. Stretching the catheter body may increase the flexibility of the catheter body. In some embodiments, the catheter body is stretched to a desired length and/or flexibility. In some embodiments, the catheter body is stretched to a desired degree of stretch. In some embodiments, the first and second layers are laminated to the catheter body while the catheter body is being stretched. In some embodiments, the stretched catheter body is held to a desired degree of stretch while the first and second layers are laminated to the catheter body. In some embodiments, laminating the first and second layers to the stretched catheter fixes the degree of stretch for the laminated portion of the catheter. In some embodiments, the stretched catheter body is actively stretched while the first and second layers are laminated to the catheter body. This may allow the stretch at the first end of the catheter to be less than the stretch at the second end of the catheter. In some embodiments, the first end of the catheter is less flexible than the second end of the catheter. In some embodiments, lamination the catheter body comprises laminating a first section of the stretched catheter having a first degree of stretch to fix the degree of stretch of the first section. After the first section is fixed, increase or decrease the degree of stretch of an unlaminated second portion of the stretched catheter and laminate the second section to fix the degree of stretch of the second section. This may allow for the different sections of the catheter to have different flexibility and/or stiffness properties.

### Connection Joint

In some embodiments, the catheter body comprises a single material. In some embodiments, the catheter body is formed from a single tube. In some embodiments, the catheter body is formed from a hypotube. In some embodiments, the catheter body has a consistent diameter along the length of the catheter body. In some embodiments, the catheter body has a first section comprising a first diameter and a second section comprising a second diameter. In some embodiments, the first section is a proximal and the second section is a distal section. In some embodiments, the first diameter is larger than the second diameter. In some embodiments, the first diameter is smaller than the second diameter. In some embodiments, the catheter body has a variable diameter along the length of the catheter body. In some embodiments, the catheter body has one or more transitioning sections between a first diameter and a second diameter. In some embodiments, the catheter body has a one or more transition section between one or more changes in the diameter of the catheter body.

In some embodiments, the lumen of the catheter has a consistent diameter along the length of the catheter. In some embodiments, the lumen has a first section comprising a first diameter and a second section comprising a second diameter. In some embodiments, the first section is a proximal and the second section is a distal section. In some embodiments, the first diameter is larger than the second diameter. In some embodiments, the first diameter is smaller than the second diameter. In some embodiments, the catheter lumen has a variable diameter along the length of the catheter. In some embodiments, the lumen has one or more transitioning sections between a first diameter and a second diameter. In some embodiments, the lumen has a one or more transition section between one or more changes in the diameter of the catheter. In some embodiments, the diameter of the catheter body does not change with a change in the lumen diameter. In some embodiments, the diameter of the catheter body increases when the lumen diameter increases. In some embodiments, the diameter of the catheter body decreases when the lumen diameter decreases.

In some embodiments, the lumen of the catheter body is between about 0.01 inches (in) to about 0.2 in. In some embodiments, the lumen of the catheter body is between about 0.01 inches (in) to about 0.1 in. In some embodiments, the lumen of the catheter body is smaller than 0.1 in. In some embodiments, the lumen of the catheter body is larger than 0.01 in. In some embodiments, the lumen of the catheter body is at least about 0.01 in, about 0.02 in, about 0.03 in, about 0.04 in, about 0.05 in, about 0.06 in, about 0.07 in, about 0.08 in, about 0.09 in, about 0.1 in, about 0.11 in, about 0.12 in, about 0.13 in, about 0.14 in, about 0.15 in, about 0.16 in, about 0.17 in, about 0.18 in, about 0.19 in, or about 0.2 in. In some embodiments, the lumen of the catheter body is at most about 0.01 in, about 0.02 in, about 0.03 in, about 0.04 in, about 0.05 in, about 0.06 in, about 0.07 in, about 0.08 in, about 0.09 in, about 0.10 in, about 0.11 in, about 0.12 in, about 0.13 in, about 0.14 in, about 0.15 in, about 0.16 in, about 0.17 in, about 0.18 in, about 0.19 in, or about 0.2 in. In some embodiments, the catheter has a diameter in the range of about 1 French (Fr) from about 20 Fr. In some embodiments, the catheter has a diameter in the range of about 1 Fr to about 10 Fr. In some embodiments, the catheter has a diameter of about 1 Fr, 1.5 Fr, 2 Fr, 2.5 Fr, 3 Fr, 3.5 Fr, 4 Fr, 4.5 Fr, 5 Fr, 5.5 Fr, 6 Fr, 6.5 Fr, 7 Fr, 7.5 Fr, 8 Fr, 8.5 Fr, 9 Fr, 9.5 Fr, 10 Fr, 10.5 Fr, 11 Fr, 11.5 Fr, 12 Fr, 12.5 Fr, 13 Fr, 13.5 Fr, 14 Fr, 14.5 Fr, 15 Fr, 15.5 Fr, 16 Fr, 16.5 Fr, 17 Fr, 17.5 Fr, 18 Fr, 18.5 Fr, 19 Fr, 19.5 Fr, or 20 Fr.

In some embodiments, a catheter as described here is configured to be a variable stiffness catheter. In some embodiments, the variable stiffness catheter comprises at least a first catheter body and a second catheter body. In some embodiments, the first catheter body and the second catheter body may be releasably coupled. In some embodiments, the first catheter body and the second catheter body may be fixedly coupled. In some embodiments, the first catheter body and the second catheter body are coupled to form a single tube structure. In some embodiments, the first catheter body and the second catheter body are coupled at a connection joint. In some embodiments, the second catheter body is proximal to the first catheter body. In some embodiments, the proximal second catheter body is stiffer than the distal first catheter body. In some embodiments, the proximal second catheter body comprises stainless steel. In some embodiments, the distal first catheter body comprises NiTi.

Sometimes, the cost to manufacture may be a significant challenge to adopting cut tubes, especially laser cut tubes, in catheters. As such, the material for a cut tube should be chosen carefully, based on the intended function of the catheter. In some cases, using a cut tube that is stainless steel for its entire length is a good choice due to its relatively low cost. In cases where the cut tube structure needs to deform more than what stainless steel can safely provide, it may be better to use superelastic NiTi alloy due to its larger range of superelastic deformation. While superelastic NiTi has good superelastic deformation properties, superelastic NiTi may be expensive. In case cases, such catheter having joined catheter bodies strikes a balance between cost and performance by forming a hybrid catheter body.

In some embodiments, a variable stiffness catheter comprises a hybrid catheter body. The hybrid catheter body may combine two or more catheter bodies. In some embodiments, the two or more catheter bodies are made of different materials. In some embodiment, a catheter body may comprise a first catheter body and a second catheter body. In some embodiments, the first catheter body may comprise stainless steel. The first catheter body may be used when high levels of deformation are not expected in that region. In some embodiments, the distal end of the second catheter body comprises a superelastic NiTi. In some embodiments, the second catheter body may be used when high levels of deformation are expected.

In some embodiments, a hybrid catheter body comprises a first catheter body and a second catheter body coupled at connection joint. In some embodiments, the first catheter body is formed from a first material. In some embodiments, the second catheter body is formed from a second material. In some embodiments, the second catheter body is stiffer than the first catheter body. In some embodiments, the second catheter body comprises stainless steel. In some embodiments, the first catheter body comprises NiTi. In some embodiments, the second catheter body is coupled to the first catheter body by a joint connection section. In some embodiments, it is beneficial to minimize the length of the joint connection section of the catheter.

In some embodiments, the stiffness of the second catheter body is variable along the length of at least a portion of the second catheter body. In some embodiments, a proximal end of the second catheter body is stiffer than the distal end of the second catheter body. In some embodiments, the flexibility of the second catheter body is variable along a length of the second catheter body. In some embodiments, a distal end of the second catheter body is more flexible than a proximal end of the second catheter body. In some embodiments, a stiffness of the first catheter body is variable along a length of at least a portion the first catheter body. In some embodiments, a proximal end of the first catheter body is stiffer than a distal end of the first catheter body. In some embodiments, the flexibility of the first catheter body is variable along a length of the first catheter body. In some embodiments, a distal end of the first catheter body is more flexible than a proximal end of the first catheter body. In some embodiments, a proximal section of the catheter body has less axial flexibility than a distal section of the catheter body.

In some embodiments, a catheter body comprises a length from about 80 centimeters (cm) to about 200 cm. In some embodiment, the catheter body comprises a length from about 80 cm to about 200 cm, or about 90 cm to about 200 cm, about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiment, the catheter body comprises a length of about 120 cm, about 130 cm, about 140 cm, about 150 cm, about 160 cm, about 170 cm, about 180 cm, about 190 cm, or about 200 cm. In some embodiment, the catheter body comprises a length of at least about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiment, the catheter body comprises a length at most about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm.

In some embodiments, a catheter body may be a hybrid catheter. In some embodiments, the hybrid catheter comprises one or more catheter bodies. The one or more catheter bodies may each or in combination, comprises a length from about 10 centimeters (cm) to about 200 cm. In some embodiments, one or more of the catheter bodies comprise a length from about 10 centimeters (cm) to about 200 cm In some embodiments, one or more of the catheter bodies comprise a length from about 10 cm to about 200 cm, about 20 cm to about 200 cm, about 20 cm to about 200 cm, about 20 cm to about 200 cm, about 40 cm to about 200 cm, about 50 cm to about 200 cm, about 60 cm to about 200 cm, about 70 cm to about 200 cm, about 80 cm to about 200 cm, about 90 cm to about 200 cm, or about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiments, one or more of the catheter bodies comprise a length of about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, one or more of the catheter bodies comprise a length of at least about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiments, one or more of the catheter bodies comprise a length at most about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, at least one of the one or more catheter bodies comprise stainless steel. In some embodiments, at least one of the one or more catheter bodies comprise NiTi. In some embodiments, one or more of the catheter bodies comprise a first catheter body and a second catheter body.

In some embodiments, a first catheter body comprises a length from about 10 centimeters (cm) to about 200 cm. In some embodiment, a first catheter body comprises a length from about 10 cm to about 200 cm, about 20 cm to about 200 cm, about 20 cm to about 200 cm, about 20 cm to about 200 cm, about 40 cm to about 200 cm, about 50 cm to about 200 cm, about 60 cm to about 200 cm, about 70 cm to about 200 cm, about 80 cm to about 200 cm, about 90 cm to about 200 cm, about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiment, a first catheter body comprises a length of about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiment, a first catheter body comprises a length of at least about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiment, a first catheter body comprises a length at most about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, the first catheter body comprises stainless steel. In some embodiments, the first catheter body comprises NiTi.

In some embodiments, a second catheter body comprises a length from about 10 centimeters (cm) to about 200 cm. In some embodiment, a second catheter body comprises a length from about 10 cm to about 200 cm, about 20 cm to about 200 cm, about 20 cm to about 200 cm, about 20 cm to about 200 cm, about 40 cm to about 200 cm, about 50 cm to about 200 cm, about 60 cm to about 200 cm, about 70 cm to about 200 cm, about 80 cm to about 200 cm, about 90 cm to about 200 cm, about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiment, a second catheter body comprises a length of about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiment, a second catheter body comprises a length of at least about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiment, a second catheter body comprises a length at most about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, the second catheter body comprises stainless steel. In some embodiments, the second catheter body comprises NiTi.

In some embodiments, the joint connection section may comprise a proximal joint connection section coupled to the first catheter body and a distal joint connection section coupled to the second catheter body. In some embodiments, the second catheter body and the distal joint connection section are formed from a single material. In some embodiments, the first catheter body and the proximal joint connection section are formed from a single material. In some embodiments, the joint connection section is formed using a cutting element. In some embodiments, the joint connection section comprises one or more joint features, configured to couple the second catheter body to the first catheter body. In some embodiments, the joint feature may comprise a receiver element and an insertion element, wherein the receiver element is configured to receive and securely hold the insertion element forming a joint. In some embodiments, the joint feature may comprise a pair of prongs configured to receive and securely hold a post. The post may be configured to form a snap fit pair junction with the pair of prongs. The post may comprise a flared tip configured to engage with teeth of the pair of prongs once inserted, such that the posts cannot be pulled out of the joint. In some embodiments, a welding pad extends between the pair of prongs. In some embodiments, the welding pad may be configured to extend a distance between the prongs such that the tip of the welding pad is adjacent to and/or in contact with the flared tip of the post while the post is engaged with the prongs. In some embodiments, the post is bonded to the welding pad. The post may be bonded to the welding pad using welding, adhesive, soldering or a combination thereof. In some embodiments, the joint feature is configured so that the post and the prongs can be snapped together quickly and accurately during assembly to reduce assembly cost and improve quality of the finished catheter. To ensure permanent fixation of the first and second catheter bodies, a laser weld, solder, or other joining techniques can be used to ensure the first and second catheter bodies stay attached to each other during use. In some embodiments, the length of the second catheter body is greater than the length of the first catheter body. In some embodiments, the length of the second catheter body is less than the length of the first catheter body. In some embodiments, the length of the second catheter body is equal to the length of the first catheter body.

In some embodiments, the joint connection section comprises an inner liner. In some embodiments, the joint connection section comprises an outer jacket. In some embodiments, the joint connection section does not have one or more of the inner liner or the outer jacket. In some embodiments, the connection section does not comprise a cut filler. In some embodiments, the connection section comprises a cut filler. In some embodiments, the lamination of the catheter layers may be configured to form an additional coupling element of the joint connection section. In some embodiments, the joint connection section comprise three coupling forces configured to prevent movement and/or separation of the second catheter body and first catheter body, 1) The engagement between the receiver (e.g., a pair of prongs) and the insertion element (e.g., a post); 2) the bonding element coupling the welding pad and the tip of the post (i.e., welded connection, soldered connection); and 3) the lamination of the catheter body at the joint connection section. In some embodiments, the catheter body may comprise more than two catheter bodies coupled together. In some embodiments, adjacent catheter bodies are coupled by a connection joint. In some embodiments, a first pair of catheter bodies are couple using a first connection joint and a second pair of catheter bodies are couple using a second connection joint. In some embodiments, the first connection joint has a first configuration, and the second connection joint has a second configuration. In some embodiments, the first connection joint connection is different than the second connection joint configuration. In some embodiments, the first connection point is proximal the second connection joint. In some embodiments, the first connection joint has a length shorter than the length of the second connection joint. In some embodiments, the first connection joint has a length longer than the length of the second connection joint. In some embodiments, the length of a connection joints between catheter bodies is equal to or less than a proximal connection joint.

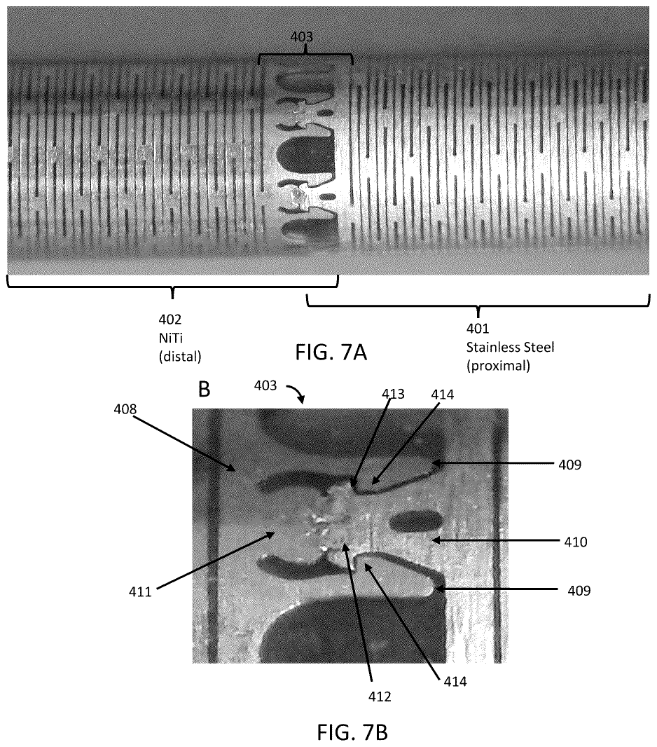

FIGS. 7A-7B illustrates an embodiment of a variable stiffness catheter having a first catheter body and a second catheter body that are joined. In comes cases, such catheter having joined catheter bodies strikes a balance between cost and performance by forming a hybrid catheter body. In some embodiments, the hybrid catheter body may combine two catheter bodies made of different materials. In some embodiment, a catheter body may comprise a first catheter body 401 and a second catheter body 402. In some embodiments, the first catheter body may comprise stainless steel. The first catheter body may be used when high levels of deformation are not expected in that region. In some embodiments, the distal end of the second catheter body 402 comprises a superelastic NiTi. In some embodiments, the second catheter body may be used when high levels of deformation are expected.

FIG. 7A illustrates an embodiment of a hybrid catheter body. In some embodiments, a catheter body comprises a first catheter body 401 and a second catheter body 402 coupled at connection joint. In some embodiments, the second catheter body is formed from a first material. In some embodiments, the first catheter body is formed from a second material. In some embodiments, the second catheter body is stiffer than the first catheter body. In some embodiments, the second catheter body comprises stainless steel. In some embodiments, the first catheter body comprises NiTi. In some embodiments, the second catheter body is coupled to the first catheter body by a joint connection section 403. In some embodiments, it is beneficial to minimize the length of the joint connection section 403 of the catheter.

In some embodiments, the stiffness of the second catheter body is variable along the length of the first section. In some embodiments, a proximal end of the second catheter body is stiffer than the distal end of the second catheter body. In some embodiments, the flexibility of the second catheter body is variable along a length of the second catheter body. In some embodiments, a distal end of the second catheter body is more flexible than a proximal end of the second catheter body. In some embodiments, the stiffness of the first catheter body is variable along a length of the distal section. In some embodiments, a proximal end of the first catheter body is stiffer than a distal end of the first catheter body. In some embodiments, the flexibility of the first catheter body is variable along a length of the first catheter body. In some embodiments, a distal end of the first catheter body is more flexible than a proximal end of the first catheter body. In some embodiments, a proximal section of the catheter body has less axial flexibility than a distal section of the catheter body.

In some embodiments, the joint connection section 403 may comprise a proximal joint connection section coupled to the first catheter body and a distal joint connection section coupled to the second catheter body. In some embodiments, the second catheter body and the distal joint connection section are formed from a single material. In some embodiments, the first catheter body and the proximal joint connection section are formed from a single material. In some embodiments, the joint connection section is formed using laser cutting. In some embodiments, the joint connection section comprises one or more joint features 408, configured to couple the second catheter body to the first catheter body. FIG. 7B illustrates an example of a joint feature configuration for coupling the second catheter body and the first catheter body. In some embodiments, the joint feature 408 may comprise a receiver element and an insertion element, wherein the receiver element is configured to receive and securely hold the insertion element forming a joint. In some embodiments, the joint feature 408 may comprise a pair of prongs 409 configured to receive and securely hold a post 410. The post 410 may be configured to form a snap fit pair junction with the pair of prongs 409. The post 410 may comprise a flared tip 413 configured to engage with teeth 414 of the pair of prongs once inserted, such that the posts cannot be pulled out of the joint. In some embodiments, a welding pad 411 extends between the pair of prongs. In some embodiments, the welding pad may be configured to extend a distance between the prongs 409 such that the tip of the welding pad is adjacent to and/or in contact with the flared tip 413 of the post 410 while the post is engaged with the prongs 409. In some embodiments, the post 410 is bonded to the welding pad 411. The post 410 may be bonded to the welding pad 411 using welding, adhesive, soldering or a combination thereof. In some embodiments, the joint feature 408 is configured so that the post 410 and the prongs 409 can be snapped together quickly and accurately during assembly to reduce assembly cost and improve quality of the finished catheter. To ensure permanent fixation of the first and second catheter bodies, a laser weld 412, solder, or other joining techniques can be used to ensure the first and second catheter bodies stay attached to each other during use. In some embodiments, the length of the second catheter body is greater than the length of the first catheter body. In some embodiments, the length of the second catheter body is less than the length of the first catheter body. In some embodiments, the length of the second catheter body is equal to the length of the first catheter body.

In some embodiments, the joint connection section comprises an inner liner. In some embodiments, the joint connection section comprises an outer jacket. In some embodiments, the joint connection section does not have one or more of the inner liner or the outer jacket. In some embodiments, the connection section does not comprise a cut filler. In some embodiments, the connection section comprises a cut filler. In some embodiments, the lamination of the catheter layers may be configured to form an additional coupling element of the joint connection section. In some embodiments, the joint connection section comprise three coupling forces configured to prevent movement and/or separation of the second catheter body and first catheter body, 1) The engagement between the receiver (e.g., the pair of prongs 409) and the insertion element (e.g., the post 410); 2) the bonding element coupling the welding pad 411 and the tip of the post 410 (i.e., welded connection, soldered connection); and 3) the lamination of the catheter body at the joint connection section. In some embodiments, the catheter body may comprise more than two catheter bodies coupled together.

In some embodiments the joint connection section comprises a length from about 0.1 centimeters (cm) to about 5 cm. In some embodiment, a second catheter body comprises a length from about 0.5 cm to about 5 cm, about 1 cm to about 5 cm, about 1.5 cm to about 5 cm, about 2 cm to about 5 cm, about 2.5 cm to about 5 cm, about 3 cm to about 5 cm, about 3.5 cm to about 5 cm, about 4 cm to about 5 cm, or about 4.5 cm to about 5 cm. In some embodiments, the joint connection section comprises a length of at least about 0.5 cm, about 1.5 cm, about 2 cm, about 2.5 cm, about 3 cm, about 3.5 cm, about 4 cm, about 4.5 cm, or about 5 cm. In some embodiments, the joint connection section comprises a length of at most about 0.5 cm, about 1.5 cm, about 2 cm, about 2.5 cm, about 3 cm, about 3.5 cm, about 4 cm, about 4.5 cm, or about 5 cm.

In some embodiment, the joint connection section comprises a length from about 0.1 cm to about 2 cm, about 0.2 cm to about 2 cm, about 0.3 cm to about 2 cm, about 0.4 cm to about 2 cm, about 0.50 cm to about 2 cm, about 0.6 cm to about 2 cm, about 0.7 cm to about 2 cm, about 0.8 cm to about 2 cm, or about 0.9 cm to about 2 cm, about 1 cm to about 2 cm, about 1.1 cm to about 2 cm, about 1.2 cm to about 2 cm, about 1.3 cm to about 2 cm, about 1.4 cm to about 2 cm, about 1.5 cm to about 2 cm, about 1.6 cm to about 2 cm, about 1.70 cm to about 2 cm, about 1.9 cm to about 2 cm, or about 1.9 cm to about 2 cm. In some embodiments, the joint connection section comprises a length of about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.1 cm, about 1.2 cm, about 1.3 cm, about 1.40 cm, about 1.5 cm, about 1.6 cm, about 1.7 cm, about 1.8 cm, about 1.9 cm, or about 2 cm. In some embodiments, the joint connection section comprises a length of at least about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, or about 2 cm. In some embodiments, the joint connection section comprises a length at most about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, or about 2 cm. In some embodiments, the joint connection section comprises stainless steel, NiTi, or a combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element is present or not (for example, detection). These terms can include quantitative, qualitative, or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be endometriosis. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

In some embodiments, flexibility refers to the ability of a material to deform elastically and return to its original shape when the applied stress is removed. In some embodiments, flexibility refers to the ability of a material to deform or bend without breaking. In some embodiments, a more flexible material may allow for a material to navigate through curved or tortuous pathways. In some embodiments, flexibility may be measured in terms of the catheter's bending radius, ability to recover its original form after deformation, and/or resistance to kinking under various conditions. In some embodiments, the flexibility may be measured as yield strength, elastic modulus, and/or flexural modulus. In some embodiments, the flexibility may be measured by a durometer.

In some embodiments, stiffness refers to the extent to which a material resists deformation in response to an applied force. In some embodiments, stiffness may refer to hardness of the material. In some embodiments, the flexibility may be measured as elastic modulus, Young's modulus, and/or tensile modulus. In some embodiments, stiffness is calculated as a ratio of force to deflection.

The section headings used herein are for organizational purposes only and are not to be construed as limiting the subject matter described.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Numbered Embodiments

Provided herein are following exemplary embodiments:
1. A catheter system for embolizing a middle meningeal artery (MMA), the system comprising:
   an exterior catheter having a lumen; and
   an interior catheter comprising a laser cut tube (LCT), the interior catheter configured to extend within the lumen of the exterior catheter,
   wherein the exterior catheter has an outer diameter at a distal portion of at most about 0.04 inch,
   wherein the interior catheter has an outer diameter at a distal portion of at most about 0.03 inch, and
   wherein a ratio of the outer diameter at the distal portion of the interior catheter to an outer diameter of a proximal portion of the interior catheter is between about 0.8 and about 1.2.
2. A catheter system for embolizing a middle meningeal artery (MMA), the system comprising:
   an exterior catheter having a lumen; and
   an interior catheter comprising a laser cut tube (LCT) having a lumen, the interior catheter configured to extend within the lumen of the exterior catheter,
   wherein the exterior catheter is configured to fit within the MMA,
   wherein the outer diameter of the interior catheter varies at most by 10% longitudinally.
3. A catheter system for delivering an embolic composition to a blood vessel, the system comprising:
   an exterior catheter comprising a laser cut tube (LCT) having a lumen; and
   an interior catheter comprising an LCT, the interior catheter configured to extend within the lumen of the exterior catheter.
4. The catheter system of embodiment 1 or 2, wherein the exterior catheter comprises an LCT.
5. The catheter system of embodiment 1 or 3, wherein the exterior catheter is configured to fit within the MMA.
6. The catheter system of embodiment 1 or 3, wherein the outer diameter of the interior catheter varies by at most 10% longitudinally.
7. The catheter system of embodiment 2 or 3, wherein the exterior catheter has an outer diameter at a distal portion of at most about 0.04 inch.
8. The catheter system of embodiment 2 or 3, wherein the interior catheter has an outer diameter at a distal portion of at most about 0.03 inch.
9. The catheter system of embodiment 2 or 3, wherein a ratio of the outer diameter at the distal portion of the interior catheter to an outer diameter of a proximal portion of the interior catheter is between about 0.8 and about 1.2.
10. The catheter system of any one of preceding embodiments, wherein the outer diameter of the interior catheter varies at most by 9%, 8%, 7%, 6%, 5%, 4%, 3%, 2%, or 1% longitudinally.
11. The catheter system of any one of preceding embodiments, wherein the outer diameter of the exterior catheter varies at most by 15%, 10%, 9%, 8%, 7%, 6%, 5%, 4%, 3%, 2%, or 1% longitudinally.
12. The catheter system of any one of preceding embodiments, wherein the outer diameter at a distal portion of the exterior catheter is at most about 0.035 inch, 0.03 inch, or 0.025 inch.
13. The catheter system of any one of preceding embodiments, wherein the outer diameter at the distal portion of the interior catheter is at most about 0.029 inch, 0.028 inch, 0.027 inch, 0.026 inch, 0.025 inch, 0.024 inch, 0.023 inch, 0.022 inch, 0.021 inch, or 0.02 inch.
14. The catheter system of any one of preceding embodiments, wherein a gap between the interior catheter and the exterior catheter is at least about 0.005 inch, 0.0045 inch, 0.004 inch, 0.0035 inch, 0.003 inch, 0.0025 inch, 0.002 inch, 0.0015 inch, or 0.001 inch.
15. The catheter system of embodiment 14, wherein the gap is sufficient to provide free movement of the interior catheter through the exterior catheter.
16. The catheter system of any one of preceding embodiments, wherein an inner diameter at the distal portion of the interior catheter is at least about 0.005 inch, 0.01 inch, 0.013 inch, 0.015 inch, 0.017 inch, or 0.02 inch.
17. The catheter system of embodiment 16, wherein the inner diameter at the distal portion of the interior catheter is configured to fit a guidewire.
18. The catheter system of any one of preceding embodiments, wherein the system comprises a guidewire configured to extend within a lumen of the interior catheter.
19. The catheter system of any one of preceding embodiments, wherein the interior catheter has a wall thickness that varies at most by about 5%, 10%, 15%, 20%, 25%, or 30% longitudinally.
20. The catheter system of any one of preceding embodiments, wherein the interior catheter has a wall thickness that is substantially constant.
21. The catheter system of any one of preceding embodiments, wherein the interior catheter is pliant.
22. The catheter system of any one of preceding embodiments, wherein the interior catheter has a Shore durometer of no more than about 150 A, 100 A, 90 A, 80 A, 70 A, 60 A, 50 A, 40 A, 30 A, 20 A, or 10 A.
23. The catheter system of any one of preceding embodiments, wherein the interior catheter comprises a pliant distal tip.
24. The catheter system of any one of preceding embodiments, wherein the interior catheter comprises a detachable distal tip.
25. The catheter system of any one of preceding embodiments, wherein the LCT of the interior catheter and/or the LCT of the exterior catheter comprises a first section comprising a plurality of first cuts and a cut filler comprising a polymer that is filling a first cut of the plurality of the first cut.
26. The catheter system of embodiment 25, wherein the LCT of the interior catheter and/or the LCT of the exterior catheter comprises a second section adjacent to the first section, the second section comprising a plurality of second cuts; a cut filler comprising a polymer that is filling a second cut of the plurality of second cuts, wherein the first cut of the plurality of first cuts has an average first kerf that is wider than an average kerf of the second cut of the plurality of second cuts.
27. The catheter system of embodiment 25 or 26, wherein the interior catheter and/or the exterior catheter comprises an outer surface of the interior catheter and/or the exterior catheter.
28. The catheter system of embodiment 27, wherein an outer jacket contacts and/or is bonded to the cut filler.
29. The catheter system of any one of embodiments 25-28, wherein the interior catheter and/or the exterior catheter comprises a liner covering the lumen of the interior catheter and/or the exterior catheter.
30. The catheter system of embodiment 29, wherein the liner comprises a polymer having a low coefficient of friction.
31. The catheter system of embodiment 29, wherein the liner comprises a polymer, wherein a polymer comprises polyethylene, PTFE, or poly(ether-block-amide), or a combination thereof.
32. The catheter system of any one of embodiments 29-31, wherein the liner contacts and/or is bonded to the cut filler.
33. The catheter system of any one of preceding embodiments, wherein an embolic composition comprises a liquid embolic, a particle, or a coil, or a combination thereof.
34. The catheter system of embodiment 33, wherein the liquid embolic solidifies when in contact with blood.
35. A method of delivering an embolic composition to a blood vessel, the method comprising:
   (a) advancing an exterior catheter through the vasculature proximal to a target site;
   (b) advancing an interior catheter through a lumen of the exterior catheter to the target site, wherein a distal tip of the interior catheter extends distally from a distal end of the exterior catheter; and
   (c) delivering the embolic composition through a lumen of the interior catheter to the target site.
36. The method of embodiment 35, wherein the method further comprises:
   (d) withdrawing the interior catheter from the lumen of the exterior catheter;
   (e) advancing the exterior catheter to a second target site; and
   (f) delivering the embolic composition through the lumen of the exterior catheter to the second target site.
37. The method of embodiment 35, wherein the method further comprises:
   (d) withdrawing the interior catheter from the lumen of the exterior catheter;
   (e) advancing the exterior catheter proximal to a second target site;
   (f) advancing a second interior catheter through the lumen of the exterior catheter to a second target site, wherein a distal tip of the second interior catheter extends distally from the distal end of the exterior catheter; and
   (g) delivering the embolic composition through a lumen of the second interior catheter to the second target site.
38. The method of any one of embodiments 36-37, wherein in step (e) a guidewire is positioned within the lumen of the exterior catheter to aid in advancing the exterior catheter.
39. The method of any one of embodiments 35-38, wherein the interior catheter and/or the exterior catheter is configured to advance past a bifurcation of a middle meningeal artery (MMA).
40. The method of any one of embodiments 35-39, wherein interior catheter and/or the exterior catheter is sized to fit in a distal branch of an MMA.
41. The method of any one of embodiments 35-40, wherein in step (a) the exterior catheter is advanced to a bifurcation in an MMA.
42. The method of any one of embodiments 35-41, wherein the target site and/or the second target site comprises a distal branch of an MMA.
43. The method of any one of embodiments 35-42, wherein an embolic composition comprises a liquid embolic, a particle, or a coil, or a combination thereof.
44. The method of embodiment 43, wherein the liquid embolic solidifies when in contact with blood.
45. The method of any one of embodiments 35-44, wherein an embolic composition is configured to prevent a leakage from a blood vessel at the target site and/or the second target site.
46. The method of any one of embodiments 35-45, wherein the exterior catheter comprises a laser cut tube (LCT).
47. The method of any one of embodiments 35-46, wherein the interior catheter comprises an LCT.
48. The method of any one of embodiments 35-47, wherein an outer diameter of the interior catheter varies at most by 10%, 9%, 8%, 7%, 6%, 5%, 4%, 3%, 2%, or 1% longitudinally.
49. The method of any one of embodiments 35-48, wherein the exterior catheter has an outer diameter at a distal portion of at most about 0.04 inch, 0.035 inch, 0.03 inch, or 0.025 inch.
50. The method of any one of embodiments 35-49, wherein the interior catheter has an outer diameter at a distal portion of at most about 0.03 inch, 0.029 inch, 0.028 inch, 0.027 inch, 0.026 inch, 0.025 inch, 0.024 inch, 0.023 inch, 0.022 inch, 0.021 inch, or 0.02 inch.
51. The method of any one of embodiments 35-50, wherein a ratio of the outer diameter at the distal portion of the interior catheter to an outer diameter of a proximal portion of the interior catheter is between about 0.8 and about 1.2.
52. The method of any one of embodiments 35-51, wherein the outer diameter of the exterior catheter varies at most by 15%, 10%, 9%, 8%, 7%, 6%, 5%, 4%, 3%, 2%, or 1% longitudinally.
53. The method of any one of embodiments 35-52, wherein a gap between the interior catheter and the exterior catheter is at least about 0.005 inch, 0.0045 inch, 0.004 inch, 0.0035 inch, 0.003 inch, 0.0025 inch, 0.002 inch, 0.0015 inch, or 0.001 inch.
54. The method of any one of embodiments 35-53, wherein the gap is sufficient to provide free movement of the interior catheter through the exterior catheter.
55. The method of any one of embodiments 35-54, wherein an inner diameter at the distal portion of the interior catheter is at least about 0.005 inch, 0.01 inch, 0.013 inch, 0.015 inch, 0.017 inch, or 0.02 inch.
56. The method of any one of embodiments 35-55, wherein the interior catheter has a wall thickness that varies at most by about 5%, 10%, 15%, 20%, 25%, or 30% longitudinally.
57. The method of any one of embodiments 35-56, wherein the interior catheter has a wall thickness that is substantially constant.
58. The method of any one of embodiments 35-57, wherein the interior catheter is pliant.
59. The method of any one of embodiments 35-58, wherein the interior catheter has a Shore durometer of no more than about 150 A, 100 A, 90 A, 80 A, 70 A, 60 A, 50 A, 40 A, 30 A, 20 A, or 10 A.
60. The method of any one of embodiments 35-59, wherein the interior catheter comprises a pliant distal tip.
61. The method of any one of embodiments 35-60, wherein the interior catheter comprises a detachable distal tip.

1. A catheter system for embolizing a middle meningeal artery (MMA), the system comprising:
an exterior catheter comprising
a laser cut tube (LCT) having a lumen and a plurality of first cuts, wherein the plurality of first cuts comprises a plurality of first interrupted spiral cuts, and
a cut filler comprising a polymer, wherein the polymer is configured to fill the plurality of the first cuts; and
an interior catheter comprising
a LCT having a plurality of second cuts, wherein the plurality of second cuts comprises a plurality of second interrupted spiral cuts, and
a cut filler comprising a polymer, wherein the polymer is configured to fill the plurality of second cuts, wherein the interior catheter is configured to extend within the lumen of the exterior catheter,
wherein the exterior catheter has an outer diameter at a distal portion of at most about 0.04 inch,
wherein the interior catheter has an outer diameter at a distal portion of at most about 0.03 inch, and
wherein a ratio of the outer diameter at the distal portion of the interior catheter to an outer diameter of a proximal portion of the interior catheter is between about 0.8 and about 1.2.

2. The catheter system of claim 1, wherein the outer diameter of the interior catheter varies by at most 10% longitudinally.

3. The catheter system of claim 1, wherein the system comprises a guidewire configured to extend within a lumen of the interior catheter.

4. The catheter system of claim 1, wherein the interior catheter has a wall thickness that is substantially constant.

5. The catheter system of claim 1, wherein the interior catheter comprises a flexible material.

6. The catheter system of claim 1, wherein the interior catheter comprises a distal tip comprising a flexible material.

7. The catheter system of claim 1, wherein the interior catheter comprises a detachable distal tip.

8.-10. (canceled)

11. The catheter system of claim 1, further comprising an outer jacket configured to cover an outer surface of the interior catheter and to contact the cut filler of the interior catheter, to bond to the cut filler of the interior catheter, or any combination thereof.

12. The catheter system of claim 1, further comprising an outer jacket configured to cover an outer surface of the exterior catheter and to contact the cut filler of the exterior catheter, to bond to the cut filler of the exterior catheter, or any combination thereof.

13. The catheter system of claim 1, wherein the catheter system comprises one or more liners covering at least one of a lumen of the interior catheter and the lumen of the exterior catheter.

14. The catheter system of claim 13, wherein the one or more liners comprise a liner polymer, wherein the liner polymer comprises polyethylene, PTFE, or poly(ether-block-amide), or a combination thereof.

15. The catheter system of claim 13, wherein the one or more liners are configured to contact the cut filler of the interior catheter, to bond to the cut filler of the interior catheter, to contact the cut filler of the exterior catheter, to bond to the cut filler of the exterior catheter, or any combination thereof.

16. The catheter system of claim 1, wherein the system is configured to deliver an embolic composition.

17. A catheter system for embolizing a middle meningeal artery (MMA), the system comprising:
an exterior catheter comprising
a laser cut tube (LCT) having a lumen and a plurality of first cuts, and
a cut filler comprising a polymer, wherein the polymer is configured to fill the plurality of the first cuts, wherein the plurality of first cuts comprises a plurality of first interrupted spiral cuts; and
an interior catheter comprising
a LCT having a lumen and a plurality of second cuts, wherein the plurality of second cuts comprises a plurality of second interrupted spiral cuts, and
a cut filler comprising a polymer, wherein the polymer is configured to fill the plurality of second cuts, the interior catheter configured to extend within the lumen of the exterior catheter,
wherein the exterior catheter is configured to fit within the MMA, and
wherein the outer diameter of the interior catheter varies at most by 10% longitudinally.

18.-20. (canceled)

21. The catheter system of claim 1, wherein the LCT of the exterior catheter comprises a first section comprising the plurality of first cuts, and a second section adjacent to the first section, the second section comprising a plurality of third cuts.

22. The catheter system of claim 21, wherein the plurality of first cuts has an average first kerf that is wider than an average second kerf of the plurality of third cuts.

23. The catheter system of claim 16, wherein the embolic composition comprises a liquid embolic, a particle, or a coil, or a combination thereof.
